(19) United States
(12) Patent Application Publication
(10) Pub. No.: US 2023/0227908 A1
(43) Pub. Date: Jul. 20, 2023

(54) METHODS AND BIOMARKERS FOR DIAGNOSTICS, DISEASE MONITORING, PERSONALIZED DRUG DISCOVERY AND TARGETED THERAPY OF AUTOIMMUNE DISEASE CONDITIONS

(71) Applicant: BAR-ILAN UNIVERSITY, Ramat Gan (IL)

(72) Inventors: Milana FRENKEL-MORGENSTERN, Gilon (IL); Rajesh DETROJA, Safed (IL); Vikrant PALANDE, Safed (IL); Dorith RAVIV SHAY, Michmanim (IL)

(73) Assignee: BAR-ILAN UNIVERSITY, Ramat Gan (IL)

(21) Appl. No.: 17/777,497

(22) PCT Filed: Nov. 17, 2020
(86) PCT No.: PCT/IL2020/051187

§ 371 Date: May 17, 2022

Related U.S. Application Data
(60) Provisional application No. 62/936,537, filed on Nov. 17, 2019.

(51) Int. Cl. C12Q 1/6883; C12Q 1/6869; G16B 20/00; G16H 10/40; G16H 20/00

(52) U.S. Cl. CPC C12Q 1/6883; C12Q 1/6869; G16B 20/00; G16H 10/40; G16H 20/00; C12Q 2600/156

(57) ABSTRACT

Compositions, methods and biomarkers for diagnostics, monitoring and therapy of various health and complex-disease conditions have an autoimmune component utilizing the techniques of data mining, computational biology, artificial intelligence and molecular biology are provided.

This application is the U.S. national phase of PCT Application No. PCT/IL2020/051187 filed Nov. 17, 2020, which claims the benefit of U.S. provisional application Ser. No. 62/936,537 filed Nov. 17, 2019, the disclosures of which are hereby incorporated in their entirety by reference herein.

### REFERENCE TO AN ELECTRONIC SEQUENCE LISTING

The contents of the electronic sequence listing (PCTIL2020051187-SQL Corrected MIL-P-001-US_ST25.txt; Size: 84,837 bytes; and Date of Creation: Apr. 4, 2023) is herein incorporated by reference in its entirety. No new matter is added by this incorporation.

### FIELD OF THE INVENTION

The present invention relates to compositions, methods and biomarkers for diagnostics, monitoring and therapy of various health and complex disease conditions having autoimmune component, utilizing the techniques of data mining, computational biology, artificial intelligence and molecular biology.

### BACKGROUND OF THE INVENTION

A misdirected immune response that occurs when the immune system goes awry and attacks the body itself is known as autoimmunity. Autoimmunity is present to some extent in everyone and is usually harmless. However, occasionally, it can cause a broad range of human illnesses, known collectively as autoimmune diseases. Autoimmune diseases occur when there is progression from benign autoimmunity to pathogenic autoimmunity. This progression is determined by genetic influences as well as environmental triggers. Autoimmune diseases can be classified as systemic or organ-specific depending on the extent of their clinicopathology. The systemic category includes systemic lupus erythematosus (SLE), rheumatoid arthritis (RA), scleroderma, primary Sjogren's syndrome, dermatomyositis, and systemic vasculitides. In systemic disease, autoimmunity targets ubiquitously expressed self-antigens, and end-organ injury is typically mediated by autoantibodies and, less commonly, T cells. In contrast, in organ-specific diseases, the self-antigens are typically cell- or tissue-specific in location or accessibility, and end-organ damage can be mediated by antibodies and/or T cells. Some of the more notable examples in this group, which span virtually all organ systems, include Hashimoto's thyroiditis, Graves' disease, multiple sclerosis (MS), type 1 diabetes mellitus (T1DM), anti-phospholipid (aPL) syndrome, pemphigus vulgaris, auto-immune hemolytic anemia, idiopathic thrombocytopenic purpura, psoriasis, Crohn's disease, urticaria, psoriasis, psoriatic arthritis, osteoarthritis, asthma, allergy, and myasthenia gravis. It should be emphasized, however, that although the distinction of systemic and organ-specific disorders provides a conceptual framework, the individual pathophysiology is more diverse than is implied by this simple classification. Some immune diseases can be activated by infections (bacteria, virus, drugs, chemical irritants, etc.) or other environmental factors. On the other hand, some autoimmune diseases have a genetic trend to progress which can be activated by outside intruder. There are over 80 types of autoimmune diseases. Many of them have similar symptoms which makes it hard to know if there are any possibilities of this disease. The common symptom of an autoimmune disease is inflammation, which can produce fever, fatigue, swelling, pain and redness. Symptoms for an autoimmune disease depend on what body part has been targeted.

Liquid biopsy is a newly emerging technique for diagnostics and being widely accepted because of its non-invasive approach and many advantages over biopsy-based diagnostics [1]. This technique uses circulating cell-free DNA (cfDNA) fragments and/or circulating free RNA (cfRNA). CfDNA is free floating small fragments of nucleic acids/DNA in the blood plasma that are not associated with cells or cell fragments. This cfDNA may be analyzed for specific genetic markers with varying degrees of specificity and sensitivity. Recent studies suggested that cfDNA analysis may be useful for diagnostics of various health and complex-disease conditions. In this respect, microbiome emerges as a powerful indicator of human well-being.

Microbiome is an ecological community of commensal, symbiotic, and pathogenic microorganisms that are associated with an organism. The microbiome is suspected to play at least a partial role in a number of health and complex-disease-related states, including Autoimmune diseases.

Autoimmune diseases are, and will continue to be, a mounting public health concern for the foreseeable future. For example, in the US, these chronic illnesses constitute a major component of U.S. health care spending, totaling perhaps hundreds of billions of dollars in direct and indirect costs to individual patients, insurance companies and the federal government.

Current methods and systems for analyzing the genetic markers and microbiomes of humans and providing tailor-made therapeutic means are still very limited. Therefore, there is an urgent, unmet need in game-changing technologies based on data mining and computational biology which will enable generating a characterization of the condition and generating a therapy model configured to correct the condition, thus providing tailored and personalized treatment solutions.

### SUMMARY OF THE INVENTION

The invention provides a novel powerful method for identifying, monitoring and treating a condition in a subject, wherein said condition is associated with omics-discoverable features. In one embodiment, the invention provides method for identifying a condition in a subject, wherein said condition is characterized by omics-discoverable features; the method comprising:
a. obtaining a biological sample from the at least one subject, wherein said biological sample comprises cell-free nucleic acids;
b. sequencing said cell-free nucleic acids;
c. mapping the sequencing results to the reference human genome;
d. identifying unmapped non-linear reads;
e. mapping the unmapped non-linear reads to the pre-computed sequence data set indicative of said condition; and
f. identifying at least one sequence associated with said condition.

The invention further provides a method for treating a condition in a subject, wherein said condition is characterized by omics-discoverable features, the method comprising:
a. obtaining a biological sample from at least one subject, wherein said biological sample comprises cell-free nucleic acids;
b. sequencing said cell-free nucleic acids;
c. mapping the sequencing results to the reference human genome;
d. identifying unmapped non-linear reads;
e. mapping the unmapped non-linear reads to the pre-computed sequence data set indicative of said condition;
f. identifying at least one sequence associated with said condition;
g. applying a pre-computed treatment model to identify therapeutic means suitable for treating the condition;
h. identifying the therapeutic means for treating the condition based on the pre-computed treatment model; and
i. providing the subject with the therapeutic means to thereby effectively treat the condition in the subject.

The invention further provides therapeutic means for use in the treatment of an autoimmune disease characterized by omics-discoverable features in a subject, wherein said therapeutic means are identified by applying a pre-computed treatment model designed to identify the therapeutic means suitable for treating said autoimmune disease; and, wherein said identifying of the therapeutic means comprises the steps of:
a. obtaining a biological sample from at least one subject wherein said biological sample comprises cell-free nucleic acids;
b. sequencing said cell-free nucleic acids;
c. mapping the sequencing results to the reference human genome;
d. identifying unmapped non-linear reads;
e. mapping the unmapped non-linear reads to the pre-computed sequence data set indicative of said condition;
f. identifying sequences associated with said condition; and,
g. applying a pre-computed treatment model to identify therapeutic means suitable for treating the condition.

The invention further provides an isolated nucleotide sequence having at least 75% sequence identity to a nucleotide sequence selected from the group consisting of SEQ ID NO:1-17.

The invention further provides an isolated nucleotide sequence having at least 75% sequence identity to a nucleotide sequence selected from the group consisting of SEQ ID NO:18-38.

The invention yet further provides an isolated nucleotide sequence selected from the group consisting of nucleotide sequences set forth as SEQ ID NO: 1-38.

### BRIEF DESCRIPTION OF THE DRAWINGS

Exemplary non-limited embodiments of the disclosed subject matter will be described, with reference to the following description of the embodiments, in conjunction with the figures. The figures are generally not shown to scale and any sizes are only meant to be exemplary and not necessarily limiting, corresponding or like elements are optionally designated by the same numerals or letters.

FIG. 1 is a graphic illustration of an exemplary embodiment of method for treating a condition in a human subject, wherein said condition is characterized by with the omics-discoverable features; and

FIG. 2(A-D) presents a list of bacteria, viruses, worms and other parasites found in the circulating cell free DNA samples of 12 patients as additional omics-derived sequence features.

### DETAILED DESCRIPTION OF THE INVENTION

The present invention is now described more fully hereinafter. This invention may, however, be embodied in many different forms and should not be construed as limited to the embodiments set forth herein; rather these embodiments are provided so that this disclosure will be thorough and complete and will fully convey the scope of the invention to those skilled in the art. In one embodiment of the invention, provided a method for identifying a condition in a subject, wherein said condition is characterized by omics-discoverable features; the method comprising:
a. obtaining a biological sample from the at least one subject, wherein said biological sample comprises cell-free nucleic acids;
b. sequencing said cell-free nucleic acids;
c. mapping the sequencing results to the reference human genome;
d. identifying unmapped non-linear reads;
e. mapping the unmapped non-linear reads to the pre-computed sequence data set indicative of said condition; and
f. identifying at least one sequence associated with said condition.

In one embodiment, the method of the invention further includes a step of isolating circulating cell-free nucleic acids from the biological sample. In the context of the invention, the phrase “a method of identifying a condition” is meant to be understood, without limitation, as diagnostic means, namely a method for diagnosing, recognizing, detecting and monitoring various characteristics of a certain disease or a condition.

As used herein, the phrase “pre-computed sequence data” refers, without limitation, to a pre-computed set of nucleotide sequences found in databases or generated using some heuristic or combination as random sequences; or merged as parts of sequences to a set of novel sequences, including separated and merged exons, introns, genes, pseudogenes or any genomic sequences and/or chimeric RNA sequences or fusion genes sequences that cannot be mapped to human genome linearly; and genomic “integrations” of pathogens to human genome. As used herein the term “omics” refers, without limitation to genomics, proteomics, metagenomics, methylomics, epigenomics, and metabolomics. As used herein the term “biological sample” refers, without limitation to any biological material collected from a subject. A non-limiting list of biological samples of the invention includes blood, serum, plasma, urine, saliva, amniotic fluid, feces, synovial fluid, peritoneal fluid, pleural fluid, lymphatic fluid, mucus, and cerebrospinal fluid (CSF), or any other body fluid or acceptable body tissue, such as biopsy. In one embodiment, the biological sample is a liquid biological sample. As used herein, the term “circulating cell-free nucleic acids” refers, without limitation to degraded nucleic acid fragments released to the blood plasma or other body fluids. In one embodiment, the circulating cell-free nucleic acids are selected from the group consisting of circulating cell-free RNA, circulating cell-free nucleic acid complexes, circulating cell-free DNA, circulating cell-free microRNA. In another embodiment, the circulating cell-free nucleic acids is circulating cell-free DNA. As used herein, the term “sequence” refers, without limitation to oligonucleotide or polynucleotide. As used herein, the terms “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs with or without protein complexes. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene”, “allele” or “gene sequence” is used broadly to refer to a DNA (deoxynucleic nucleic acids) associated with a biological function. Thus, genes may include introns and exons as in the genomic sequence or may comprise only a coding sequence as in cDNAs, and/or may include cDNAs in combination with regulatory sequences. Thus, according to the various aspects of the invention, genomic DNA, cDNA or coding DNA may be used.

In one embodiment of the invention, the condition associated with omics-discoverable features is an autoimmune disorder. In another embodiment, the autoimmune disorder is selected from the group consisting of systemic lupus erythematosus (SLE), rheumatoid arthritis (RA), scleroderma, primary Sjogren's syndrome, dermatomyositis, systemic vasculitides, Hashimoto's thyroiditis, Graves' disease, multiple sclerosis (MS), type 1 diabetes mellitus (T1DM), anti-phospholipid (aPL) syndrome, pemphigus vulgaris, auto-immune hemolytic anemia, idiopathic thrombocytopenic purpura, myasthenia gravis, Chronic inflammatory demyelinating polyneuropathy, Guillain-Barre syndrome, psoriasis, Crohn's disease, urticaria, psoriasis, psoriatic arthritis, osteoarthritis, asthma, allergy, and Inflammatory Bowel disease (IBD). In one embodiment, the autoimmune disorder is systemic lupus erythematosus (SLE). In one embodiment, the autoimmune disorder is rheumatoid arthritis (RA). As used herein, the term “autoimmune disorder” refers, without limitation to any condition arising from an aberrant immune response of an organism against its own healthy cells and tissues. In the context of the invention, the autoimmune disease can be, without limitation, a newly diagnosed autoimmune disease, an ongoing active autoimmune disease, a progressive autoimmune disease, an autoimmune disease under remission, an autoimmune disease accompanying another non-autoimmune disease state, or a combination of more than one autoimmune disease.

In one embodiment of the invention, the sequence associated with said condition is a gene fusion. The term “gene fusion” refers to a chimeric genomic DNA resulting from the fusion of at least a portion of a first gene to a portion of a second gene and exon-intron or exon-exon or intron-exon “non-linear” combinations. The point of transition between the sequences from the first gene in the fusion to the sequences from the second gene in the fusion is referred to as the “breakpoint” or “fusion point” and/or “chimeric junction site”. Transcription of the gene fusion results in a chimeric mRNA and/or chimeric RNA transcript. As used herein in, the term “chimeric RNA transcript” refers, without limitation, to single-stranded sequences of RNAs transcribed from various locations in the total genome corresponding to exons and/or introns from two different genes and/or non-linear exons-introns combinations of the same gene; two copies of the same gene; regions of pathogen genome, which fuse together to produce a single RNA transcript and/or a single cell free DNA molecule. Two unrelated genomic loci on different chromosomes may produce a chimeric transcript through a genomic rearrangement event or due to trans-splicing. Similarly, a read-through transcript of two adjacent genomic loci may produce chimeric RNAs. As used herein, the term “gene” refers, without limitation, to a polynucleotide (e.g., a DNA segment), that encodes a polypeptide and includes regions preceding and following the coding regions as well as intervening sequences (introns) between individual coding segments (exons).

According to some embodiments, the at least one sequence associated with the condition of the invention is selected from the group consisting of sequences having at least 75% sequence identity to the nucleotide sequence set forth as SEQ ID NO:1-17. In another embodiment, the sequence associated with said condition is selected from the group consisting of sequences having 75%-98% sequence identity to the nucleotide sequence set forth as SEQ ID NO: 1-17. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 1-17. In one embodiment, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 1-17.

According to some embodiments, the at least one sequence associated with the condition of the invention is selected from the group consisting of sequences set forth as SEQ ID NO: 1-38. According to some embodiments, the sequence associated with the condition of the invention is selected from the group consisting of sequences having at least 75% sequence identity to the nucleotide sequence set forth as SEQ ID NO: 18-38. In another embodiment, the sequence associated with said condition is selected from the group consisting of sequences having at least 75%-98% sequence identity to the nucleotide sequence set forth as SEQ ID NO: 18-38. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 18-38. In one embodiment, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 18-38. In one embodiment, the at least one sequence associated with the condition of the invention is selected from the group consisting of nucleotide sequences set forth as SEQ ID NO: 1-38.

According to some embodiments, the subject is a human subject.

According to some embodiments, the subject is a non-human subject.

As used herein, “sequence identity” or “identity” in the context of two nucleic acid sequences makes reference to the residues in the two sequences that are the same when aligned for maximum correspondence over a specified comparison window. The term further refers hereinafter to the amount of characters which match exactly between two different sequences. Hereby, gaps are not counted, and the measurement is relational to the shorter of the two sequences. It is further within the scope that the terms “similarity” and “identity” additionally refer to local homology, identifying domains that are homologous or similar (in nucleotide sequence). It is acknowledged that bioinformatics tools such as BLAST, SSEARCH, FASTA, and HMMER calculate local sequence alignments which identify the most similar region between two sequences. For domains that are found in different sequence contexts in different proteins, the alignment should be limited to the homologous domain, since the domain homology is providing the sequence similarity captured in the score. According to some aspects the term similarity or identity further includes a sequence motif, which is a nucleotide or amino-acid sequence pattern that is widespread and has, or is conjectured to have, a biological significance.

In the context of the invention, the phrase “omics-discoverable features” is meant to be understood as a characteristic that can be identified and/or recognized and/or measured by means of “omics” as defined above. In one embodiment, the omics-discoverable feature is selected from the group consisting of genomics-discoverable features, proteomics-discoverable features, metagenomics-discoverable features, methylomics-discoverable features, epigenomics-discoverable features, and metabolomics-discoverable features. The non-limiting list of omics-discoverable features of the invention include chimeras, chimeric RNAS, gene-gene fusions, sense-antisense (SAS) chimeras, exon-intron fusions, exon-exon fusions, intron-exon fusions, genomic integrations, aberrations, inversions, and other genomic alterations.

According to some embodiments, the invention provides a method for treating a condition in a subject, wherein said condition is characterized by omics-discoverable features, the method comprising:
a. obtaining a biological sample from at least one subject, wherein said biological sample comprises cell-free nucleic acids;
b. sequencing said cell-free nucleic acids;
c. mapping the sequencing results to the reference human genome;
d. identifying unmapped non-linear reads;
e. mapping the unmapped non-linear reads to the pre-computed sequence data set indicative of said condition;
f. identifying at least one sequence associated with said condition;
g. applying a pre-computed treatment model to identify therapeutic means suitable for treating the condition;
h. identifying the therapeutic means for treating the condition based on the pre-computed treatment model; and
i. providing the subject with the therapeutic means to thereby effectively treat the condition in the subject.

In one embodiment, the method further comprises the step of isolating circulating cell-free nucleic acids from the biological sample. As described herein, isolation of cell-free nucleic acids may be done by any suitable technique known in the art, commercially available or using in-house developed tools and proprietary technology. In one embodiment, the biological sample is selected from the group consisting of blood, serum, plasma, urine, saliva, amniotic fluid, feces, synovial fluid, peritoneal fluid, pleural fluid, lymphatic fluid, mucus, and cerebrospinal fluid (CSF). In another embodiment, the biological sample is a liquid biological sample. In one embodiment, the circulating cell-free nucleic acids is selected from the group consisting of circulating cell-free RNA, circulating cell-free nucleic acid complexes, circulating cell-free DNA, circulating cell-free microRNA. In one embodiment, the condition associated with omics-discoverable features is an autoimmune disorder. In one embodiment, the autoimmune disorder is selected from the group consisting of systemic lupus erythematosus (SLE), rheumatoid arthritis (RA), scleroderma, primary Sjogren's syndrome, dermatomyositis, systemic vasculitides, Hashimoto's thyroiditis, Graves' disease, multiple sclerosis (MS), type 1 diabetes mellitus (T1DM), anti-phospholipid (aPL) syndrome, pemphigus vulgaris, auto-immune hemolytic anemia, idiopathic thrombocytopenic purpura, myasthenia gravis, Chronic inflammatory demyelinating polyneuropathy, Guillain-Barre syndrome, psoriasis, Crohn's disease, urticaria, psoriasis, psoriatic arthritis, osteoarthritis, asthma, allergy, and Inflammatory Bowel disease (IBD). In one embodiment, the autoimmune disorder is systemic lupus erythematosus (SLE). In one embodiment, the autoimmune disorder is rheumatoid arthritis (RA). In one embodiment, the at least one sequence associated with the condition of the invention is selected from the group consisting of sequences having at least 75% sequence identity to the nucleotide sequence set forth as SEQ ID NO:1-17. In another embodiment, the sequence associated with said condition is selected from the group consisting of sequences having 75%-98% sequence identity to the nucleotide sequence set forth as SEQ ID NO: 1-17. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 1-17. In one embodiment, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 1-17. In another embodiment, the at least one sequence associated with the condition of the invention is selected from the group consisting of sequences set forth as SEQ ID NO: 1-38. In one embodiment, the sequence associated with the condition of the invention is selected from the group consisting of sequences having at least 75% sequence identity to the nucleotide sequence set forth as SEQ ID NO: 18-38. In another embodiment, the sequence associated with said condition is selected from the group consisting of sequences having at least 75%-98% sequence identity to the nucleotide sequence set forth as SEQ ID NO: 18-38. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 18-38. In one embodiment, the sequence associated with said condition is selected from the group consisting of sequences having at least 75°, 76°, 77°, 78°, 79°, 80°, 81°, 82°, 83°, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 18-38. In one embodiment, the at least one sequence associated with the condition of the invention is selected from the group consisting of nucleotide sequences set forth as SEQ ID NO: 1-38.

According to some embodiments, the subject is a human subject. According to some embodiments, the subject is a non-human subject.

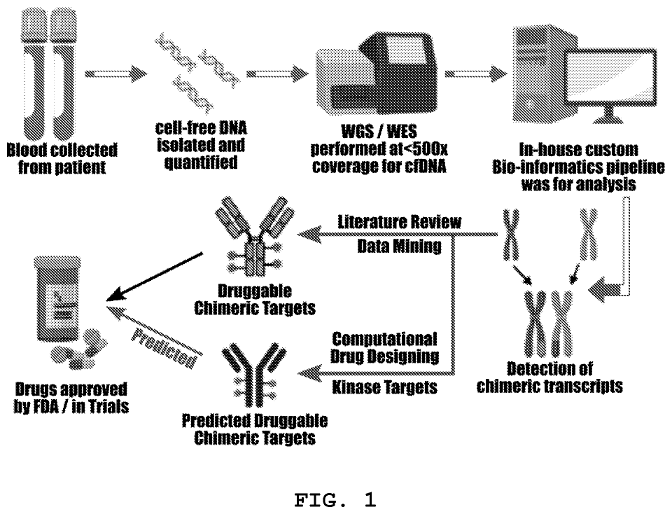

Reference is now made to FIG. 1, illustrating an exemplary embodiment of the method of the invention. As described, a biological sample in the form of blood is taken from a patient and cell-free DNA is isolated and sequenced. The sequence data are then analyzed using bioinformatics means and chimeric transcripts are detected, and data mining and computational biology are used to predict and to identify druggable targets for personalized therapy. In one embodiment, omics-discoverable features are selected from the group consisting of genomics-discoverable features, proteomics-discoverable features, metagenomics-discoverable features, methylomics-discoverable features, epigenomics-discoverable features, and metabolomics-discoverable features. In another embodiment, omics-discoverable features are selected from chimeras, chimeric RNAS, gene-gene fusions, sense-antisense (SAS) chimeras, exon-intron fusions, exon-exon fusions, intron-exon fusions, genomic integrations, aberrations, and inversions. In one embodiment, the therapeutic means are selected from the group consisting of an investigational drug, an approved drug, a food supplement, phototherapy, radiation therapy, surgical intervention, non-invasive image-guided procedure, hyperbaric oxygen, multi-step treatment protocol, or any combination thereof.

According to some embodiments the invention provides therapeutic means for use in the treatment of an autoimmune disease characterized by omics-discoverable features in a subject, wherein said therapeutic means are identified by applying a pre-computed treatment model designed to identify the therapeutic means suitable for treating said autoimmune disease; and, wherein said identifying of the therapeutic means comprises the steps of:
a. obtaining a biological sample from at least one subject wherein said biological sample comprises cell-free nucleic acids;
b. sequencing said cell-free nucleic acids;
c. mapping the sequencing results to the reference human genome;
d. identifying unmapped non-linear reads;
e. mapping the unmapped non-linear reads to the pre-computed sequence data set indicative of said condition;
f. identifying sequences associated with said condition; and,
g. applying a pre-computed treatment model to identify therapeutic means suitable for treating the condition.

In one embodiment, identifying of the therapeutic means comprises the step of isolating circulating cell-free nucleic acids from the biological sample. In one embodiment, therapeutic means are selected from the group consisting of an investigational drug, an approved drug, a food supplement, phototherapy, radiation therapy, surgical intervention, non-invasive image-guided procedure, multi-step treatment protocol, or a combination thereof. In one embodiment of the therapeutic means of the invention, the autoimmune disease is selected from the group consisting of systemic lupus erythematosus (SLE), rheumatoid arthritis (RA), scleroderma, primary Sjogren's syndrome, dermatomyositis, systemic vasculitides, Hashimoto's thyroiditis, Graves' disease, multiple sclerosis (MS), type 1 diabetes mellitus (T1DM), anti-phospholipid (aPL) syndrome, pemphigus vulgaris, auto-immune hemolytic anemia, idiopathic thrombocytopenic purpura, myasthenia gravis, Chronic inflammatory demyelinating polyneuropathy, Guillain-Barre syndrome, psoriasis, Crohn's disease, urticaria, psoriasis, psoriatic arthritis, osteoarthritis, asthma, allergy, and Inflammatory Bowel disease (IBD). In one embodiment, the therapeutic means are for use in the treatment of systemic lupus erythematosus (SLE). In one embodiment, the therapeutic means are for use in the treatment of rheumatoid arthritis (RA). In one embodiment, provided therapeutic means for use a medicament. In one embodiment of the therapeutic means of the invention, the at least one sequence associated with said autoimmune disease is isolated nucleotide sequence having at least 75% sequence identity to a nucleotide sequence selected from the group consisting of SEQ ID NO:1-38. In one embodiment of the therapeutic means of the invention, the at least one sequence associated with the condition of the invention is selected from the group consisting of sequences having at least 75% sequence identity to the nucleotide sequence set forth as SEQ ID NO:1-17. In another embodiment of the therapeutic means of the invention, the sequence associated with said condition is selected from the group consisting of sequences having 75%-98% sequence identity to the nucleotide sequence set forth as SEQ ID NO: 1-17. In another embodiment of the therapeutic means of the invention, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 1-17. In one embodiment of the therapeutic means of the invention, the sequence associated with said condition is selected from the group consisting of sequences having at least 75%, 76%, 77%, 78°, 79°, 80°, 81°, 82°, 83°, 84°, 85°, 86°, 87°, 88°, 89°, 90°, 91°, 92°, 93°, 94°, 95%, 96°, 97°, 98°, and 99° sequence identity to the nucleotide sequence set forth as SEQ ID NO: 1-17. In another embodiment of the therapeutic means of the invention, the at least one sequence associated with the condition of the invention is selected from the group consisting of sequences set forth as SEQ ID NO: 1-38. In one embodiment of the therapeutic means of the invention, the sequence associated with the condition of the invention is selected from the group consisting of sequences having at least 756 sequence identity to the nucleotide sequence set forth as SEQ ID NO: 18-38. In another embodiment of the therapeutic means of the invention, the sequence associated with said condition is selected from the group consisting of sequences having at least 75%-98% sequence identity to the nucleotide sequence set forth as SEQ ID NO: 18-38. In another embodiment of the therapeutic means of the invention, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 18-38. In one embodiment of the therapeutic means of the invention, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 18-38. In one embodiment of the therapeutic means of the invention, the at least one sequence associated with the condition of the invention is selected from the group consisting of nucleotide sequences set forth as SEQ ID NO: 1-38. In one embodiment of the therapeutic means of the invention, the biological sample is selected from the group consisting of blood, serum, plasma, urine, saliva, amniotic fluid, feces, synovial fluid, peritoneal fluid, pleural fluid, lymphatic fluid, mucus, and cerebrospinal fluid (CSF). In one embodiment of the therapeutic means of the invention, the circulating cell-free nucleic acid is selected from the group consisting of circulating cell-free RNA, circulating cell-free nucleic acid complexes, circulating cell-free DNA, circulating cell-free microRNA. In one embodiment of the therapeutic means of the invention, omics-discoverable features are selected from chimeras, chimeric RNAS, gene-gene fusions, sense-antisense (SAS) chimeras, exon-intron fusions, exon-exon fusions, intron-exon fusions, genomic integrations, aberrations, and inversions.

According to some embodiments of the therapeutic means of the invention, the subject is a human subject.

According to some embodiments of the therapeutic means of the invention, the subject is a non-human subject.

According to some embodiments, the invention provides an isolated nucleotide sequence having at least 75% sequence identity to a nucleotide sequence selected from the group consisting of SEQ ID NO:1-17. In another embodiment, the invention provides an isolated nucleotide sequence having 75%-98% sequence identity to the nucleotide sequence set forth as SEQ ID NO: 1-17. In another embodiment, the invention provides an isolated nucleotide sequence having 80%-98%, 85%-98%, 87%-98%, 90%-98%, and 95%-98% sequence identity to the nucleotide sequence set forth as SEQ ID NO: 1-17. In another embodiment, the invention provides an isolated nucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 1-17.

According to some embodiments, the invention provides an isolated nucleotide sequence selected from the group consisting of sequences set forth as SEQ ID NO: 1-17.

According to some embodiments, the invention provides an isolated nucleotide sequence having at least 75% sequence identity to a nucleotide sequence selected from the group consisting of SEQ ID NO:18-38. In another embodiment, the invention provides an isolated nucleotide sequence having 75%-98° sequence identity to the nucleotide sequence set forth as SEQ ID NO: 18-38. In another embodiment, the invention provides an isolated nucleotide sequence having 80%-98%, 85%-98%, 87%-98%, 90%-98%, and 95%-98% sequence identity to the nucleotide sequence set forth as SEQ ID NO: 18-38. In another embodiment, the invention provides an isolated nucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 18-38.

According to some embodiments, the invention provides an isolated nucleotide sequence selected from the group consisting of sequences set forth as SEQ ID NO: 18-38.

According to some embodiments, the invention provides an isolated nucleotide sequence selected from the group consisting of sequences set forth as SEQ ID NO: 1-38.

In one embodiment, the method of the invention utilizes the most comprehensive chimeric transcript repository, ChiTaRS 5.0 (http://chitars.md.biu.ac.il/), with 111,582 annotated entries from eight species. The repository includes unique information correlating chimeric breakpoints with 3D chromatin contact maps, generated from public datasets of chromosome conformation capture techniques (Hi-C). The repository comprises curated information on druggable fusion targets matched with chimeric breakpoints, which are applicable to precision medicine in cancers; as well as chimeric RNAs in various cell-lines and, novel chimeras in autoimmune diseases.ChiTaRS stands out as a unique server that integrates EST and mRNA sequences, literature resources, with RNA-sequencing data, expression level and tissue specificity of chimeric transcripts in various tissues and organisms.

According to some embodiments, the method of cfDNA analysis is based on deep Illumina sequencing procedure as well as BGI sequencing and/or other deep sequencing procedures of cfDNA and/or circulating cell free RNA (cfRNA) extracted from patients' blood plasma (using dedicated Quiagen and/or other kits), followed by the efficient Bioinformatics analysis using in-house developed tool, method and apparatus. Our method is based on the analysis of in-house collected microbiome-datasets of 32,101 viruses, 80 protozoa/worms/parasites, and 935 human bacteria species. We use the full genome mapping by means of cfDNA and/or cfRNA reads and, next, all the unmapped reads are mapped to the microbiome-dataset and/or metagenome of interest. According to some embodiments, the method for diagnosing and treating a condition associated with microbiome functional features comprises: receiving an aggregate set of samples from a population of subjects; cfDNA/cfRNA extracted and proceeded for the deep sequencing; characterizing a microbiome composition and/or functional features for each of the aggregate set of samples associated with the population of subjects, thereby generating at least one of a microbiome composition dataset and a microbiome functional diversity dataset for the population of subjects. All the deep sequencing results are mapping to the reference human genome; all the unmapped reads are used to map to the microbiome-datasets for functional diversity dataset into a characterization model of the microbiome conditions. In some variations, the first method can further include: based upon the characterization, generating a therapy model configured to improve a state of the condition. Variations of the first method can additionally or alternatively comprise clustering otherwise unrelated conditions based upon features extracted from at least one of a microbiome functional diversity dataset and/or a microbiome composition dataset by way of a characterization model; and diagnosing a subject with a condition based upon identification of microbiome functional features and/or microbiome composition features, determined upon processing a sample from the subject.

According to some embodiments, the method of the invention allows generating models that can be used to characterize and/or diagnose subjects according to at least one of their microbiome composition and functional features (e.g., as a clinical diagnostic, as a companion diagnostic, etc.), and provide therapeutic means. As used herein, the term “therapeutic means” refers, without limitation, to the remedial agents or methods for the treatment of health-disease conditions or disorders. A non-limiting list of therapeutic means of the invention includes investigational drug, an approved drug, food supplement, phototherapy, radiation therapy, immunotherapy, biological therapy, surgical intervention, non-invasive image-guided procedures, hyperbaric oxygen, multi-step treatment protocol, or any combination of the above. A non-limiting list of therapeutic means of the invention further includes probiotic-based therapeutic means, phage-based therapeutic means, small-molecule-based therapeutic means, prebiotic-based therapeutic means, clinical measures, mouth derived microbiome or any other clinically acceptable therapeutics. The therapeutic means of the invention can be, without limitation, newly discovered therapeutic means and/or known therapeutic means that are already in clinical use.

According to some embodiments, data from the population of subjects can be used to characterize subjects according to their microbiome composition and/or functional features, indicate states of health and areas of improvement based upon the characterization(s), and promote one or more therapies that can modulate the composition of a subject's microbiome toward one or more of a set of desired equilibrium states, in comparison with healthy subject.

According to some embodiments, diagnostics associated with the condition can be assessed using one or more of: a behavioral survey instrument (e.g., a Patient Health Questionnaire-9 (PHQ-9) survey, a patient health questionnaire-2 (PHQ-2) survey, a blood cell analysis of a biological sample, imaging-based method, stress testing, motion testing, biopsy, and any other standard method.

According to some embodiments, the method can be used for characterization of and/or therapeutic intervention for one or more of: anxiety, depression, Sprue, Hashimoto's Thyroiditis, chronic fatigue syndrome, arthritis, rheumatoid arthritis, psoriatic arthritis, lupus, multiple sclerosis, Crohn disease and any other associated condition. In one embodiment, the method can be used to characterize seemingly unrelated conditions, disorders, and/or adverse states in an entirely non-typical method, using microbiome-based diagnostics and/or therapeutics.

According to some embodiments, at least one of the methods is implemented, when a system receives a biological sample derived from the subject (or an environment associated with the subject) by way of a sample reception kit, and processes the biological samples (particularly, blood samples or other fluids) at a processing system implementing a characterization process and a therapy model configured to positively influence a microorganism distribution in the subject (e.g., human, non-human animals, environmental ecosystem, etc.). In variations of the application, the processing system can be configured to generate and/or improve the characterization process and the therapy model based upon sample data received from a population of subjects. The method can, however, alternatively be implemented using any other suitable system(s) configured to receive and process microbiome-related data of subjects, in aggregation with other information, in order to generate models for microbiome-derived diagnostics and associated therapeutics. Thus, the method can be implemented for a population of subjects (e.g., including the subject, excluding the subject), wherein the population of subjects can include patients dissimilar to and/or similar to the subject (e.g., in health condition, in dietary needs, in demographic features, etc.), particularly in different autoimmune conditions. Thus, information derived from the population of subjects can be used to provide additional insight into connections between behaviors of a subject and effects on the subject's microbiome, due to aggregation of data from a population of subjects.

### EXAMPLES

In the examples below, if an abbreviation is not defined above, it has its generally accepted meaning.

### Example 1

Microbiome-derived genomic integration (fusions) in the patient's genome for personalized targeted treatments.

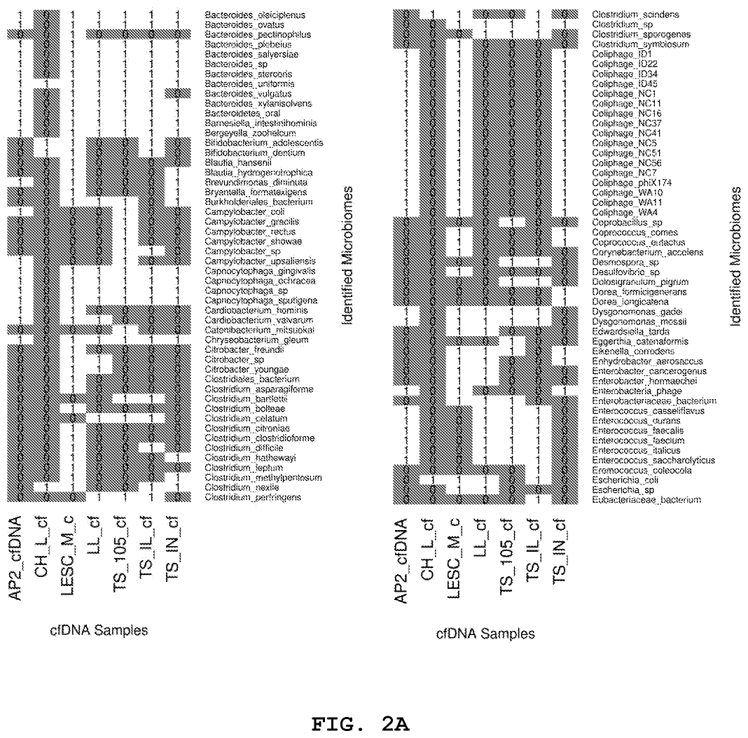

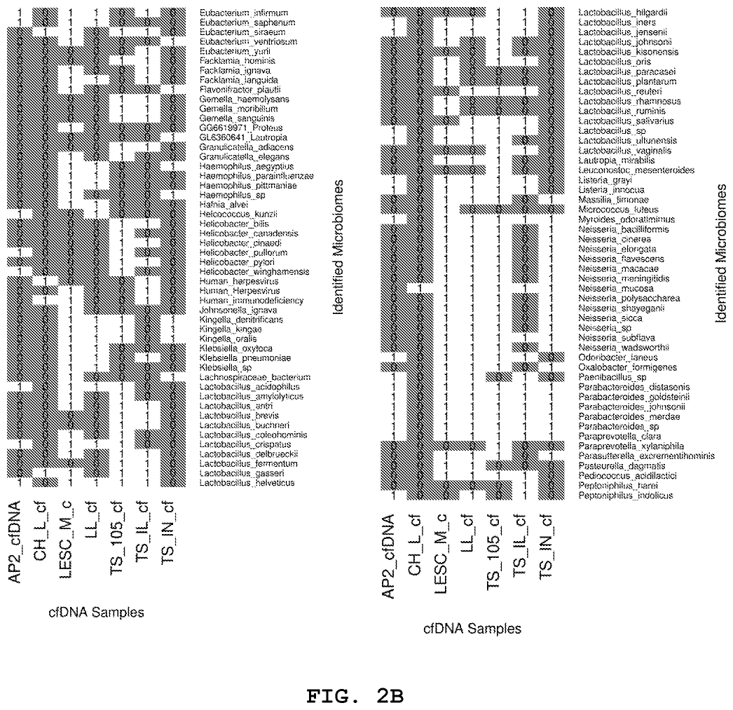

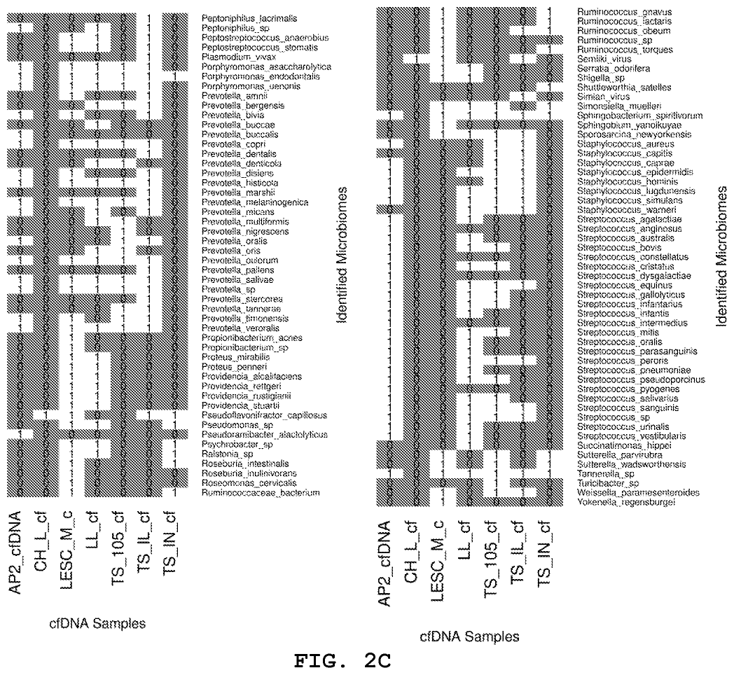

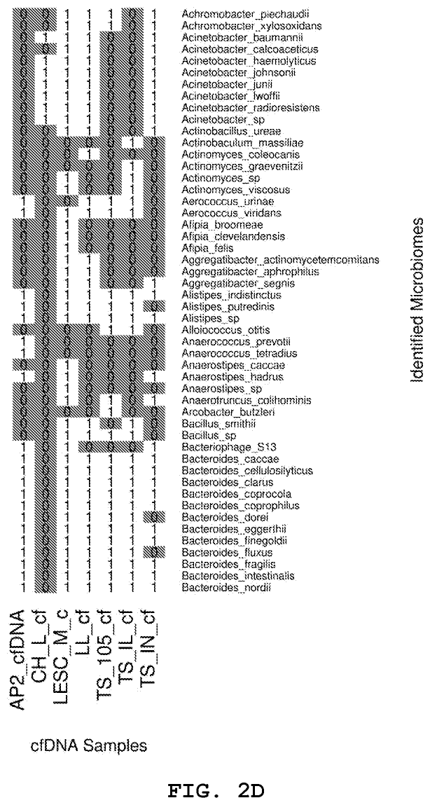

An aggregate set of samples from a population of subjects was received and cfDNA was extracted and subjected to the deep sequencing. The microbiome composition and functional features for each of the aggregate set of samples associated with the population of subjects was characterized, thereby generating at least one of: a microbiome composition dataset and a microbiome functional diversity dataset for the population of subjects. Deep sequencing results are mapping to the reference human genome; all the unmapped reads were used to map to the microbiome-datasets for functional diversity dataset into a characterization model of the microbiome conditions. Reference is now made to FIG. 2, demonstrating a list of bacteria, viruses, worms and Malaria (Plasmodium vivax) parasite found in patients' cfDNA data for 12 patients, where alterations in microbiome such than a bacteria/virus/worms/parasites is missing (0) or present (1) define their microbiome profile. Specifically, anti-malaria medicines can be used to target Plasmodium Vivax (malaria) integration into human genome. Reference is now made to Table 1, listing regions of the pathogens integrated into the patients' genome:

TABLE 1

| SEQ. ID. NO. | ORGANISM | DESCRIPTION |
|---|---|---|
| 1 | Plasmodium Vivax | comp1079_c0 |
| 2 | Plasmodium Vivax | comp11383_c1 |
| 3 | Plasmodium Vivax | comp10763_c0 |
| 4 | Plasmodium Vivax | comp33944_c0 |
| 5 | Plasmodium Vivax | comp9841_c0 |
| 6 | Plasmodium Vivax | comp86121_c0 |
| 7 | Plasmodium Vivax | comp5051_c0 |
| 8 | Plasmodium Vivax | comp725_c0 |
| 9 | Plasmodium Vivax | comp141778_c0 |
| 10 | Plasmodium Vivax | comp262756_c0 |
| 11 | Human Herpesvirus 5 | comp186508_c0 |
| 12 | Human Herpesvirus 5 | comp231082_c0 |
| 13 | Human Herpesvirus 5 | comp150744_c0 |
| 14 | Human Herpesvirus 5 | comp89065_c0 |
| 15 | Human Herpesvirus 8 | comp153629_c0 |
| 16 | Vaccinia virus | comp429_c0 |
| 17 | Simian virus | comp139079_c0 |

### Example 2

A list of the druggable genomic DNA fusions for targeted immune-therapy treatments for a subject diagnosed with autoimmune disease.

CfDNA is isolated and sequenced as previously described from biological samples of patients with autoimmune diseases. Reference is now made to Table 5 listing regions of the druggable kinase fusions (chimeras).

TABLE 2

| SEQ. ID. NO. | GROUP | DESCRIPTION |
|---|---|---|
| 18 | Predicted druggable chimeras | NUDT3/NUDT3 |
| 19 | Predicted druggable chimeras | SULF2/NCOA3 |
| 20 | Predicted druggable chimeras | BVES/EMC3 |
| 21 | Predicted druggable chimeras | CRIPT/ZFP36L1 |
| 22 | Predicted druggable chimeras | HNRNPA0/HCFC2 |
| 23 | Predicted druggable chimeras | SULT2A1/SERPINC1 |
| 24 | Predicted druggable chimeras | LENG8/RTF1 |
| 25 | Predicted druggable chimeras | LOC100996842/RCC2 |
| 26 | Predicted druggable chimeras | NEGR1/RBM3 |
| 27 | Predicted druggable chimeras | TTYH3/WDR34 |
| 28 | Predicted druggable chimeras | RACK1/ATP1A1_Junction2 |
| 29 | Predicted druggable chimeras | ANKRD52/RPS21 |
| 30 | Predicted druggable chimeras | ZFYVE16/GOT1 |
| 31 | Predicted druggable chimeras | RNA45SN1/RNA45SN2 |
| 32 | Predicted druggable chimeras | SLC25A44/RIPK2 |
| 33 | Predicted druggable chimeras | EMP2/EMP2 |
| 34 | Predicted druggable chimeras | TPX2/ERGIC1 |
| 35 | Predicted druggable chimeras | MIR44352HG/TRIOBP |
| 36 | Predicted druggable chimeras | PRB2/C1S |
| 37 | Predicted druggable chimeras | SUMF2/RILPL1 |
| 38 | Predicted druggable chimeras | PARP10/PARP10 |

Unless otherwise defined, all technical and/or scientific terms used herein have the same meaning as commonly understood by one of ordinary skill in the art, to which the invention pertains. Although methods and materials similar or equivalent to those described herein can be used in the practice or testing of embodiments of the invention, exemplary methods and/or materials are described below. In case of conflict, the patent specification, including definitions, will prevail. In addition, the materials, methods, and examples are illustrative only and are not intended to be necessarily limiting.

As used herein the terms “comprises”, “comprising”, “includes”, “including”, “having” and their conjugates mean “including but not limited to”.

The term “consisting of” means “including and limited to”.

As used herein, the singular form “a”, “an” and “the” include plural references unless the context clearly dictates otherwise. For example, the term “a compound” or “at least one compound” may include a plurality of compounds, including mixtures thereof.

It will be understood that when an element is referred to as being “on,” “attached” to, “connected” to, “coupled” with, “contacting,” etc., another element, it can be directly on, attached to, connected to, coupled with and/or contacting the other element or intervening elements can also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature can have portions that overlap or underlie the adjacent feature.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention.

Certain features described in the context of various embodiments are not to be considered essential features of those embodiments, unless the embodiment is inoperative without those elements.

As used herein, the term “non-linear reads” refers, without limitation, to nucleotide sequences which do not map linearly to the target genome. A non-limiting list of non-linear reads of the invention includes genomic integrations; exon-exon combinations; exon-intron combinations and/or any other sequence parts merged together.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

By “patient” or “subject” is meant to include any mammal. A “mammal,” as used herein, refers to any animal classified as a mammal, including but not limited to, humans, experimental animals including monkeys, rats, mice, and guinea pigs, domestic and farm animals, and zoo, sports, or pet animals, such as dogs, horses, cats, cows, and the like.

“Treating” or “treatment” of a disease as used herein includes: preventing the disease, i.e. causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms, or relieving the disease, i.e., causing regression of the disease or its clinical symptoms, and/or monitoring the disease and early diagnostics of the disease.

Druggability, is a term used in drug discovery to describe a biological target such as a protein that is known to bind or is predicted to bind with high affinity to a drug. Furthermore, the binding of the drug to a druggable target alters the function of the target with a therapeutic benefit to the patient. The term “drug” herein includes small molecules (low molecular weight organic substances) but also has been extended to include biologic medical products such as therapeutic monoclonal antibodies. In at least one embodiment, the gene fusion or gene variant can be used to identify a druggable target.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### REFERENCES

[1] Crowley, E.; Di Nicolantonio, F.; Loupakis, F.; Bardelli, A. Liquid Biopsy: Monitoring Cancer-Genetics in the Blood. Nat. Rev. Clin. Oncol. 2013, 10 (8), 472-484. https://doi.org/10.1038/nrclinonc.2013.110.
[2] Heitzer, E.; Ulz, P.; Geigl, J. B. Circulating Tumor DNA as a Liquid Biopsy for Cancer. Clin. Chem. 2015, 61 (1), 112-123. https://doi.org/10.1373/clinchem.2014.222679.
[3] Elazezy, M.; Joosse, S. A. Techniques of Using Circulating Tumor DNA as a Liquid Biopsy Component in Cancer Management. Comput. Struct. Biotechnol. J. 2018, 16, 370-378. https://doi.org/10.1016/j.csbj.2018.10.002.
[4] Snyder, M. W.; Kircher, M.; Hill, A. J.; Daza, R. M.; Shendure, J. Cell-Free DNA Comprises an In Vivo Nucleosome Footprint That Informs Its Tissues-Of-Origin. Cell 2016, 164 (1-2), 57-68. https://doi.org/10.1016/j.ce11.2015.11.050.

1-14. (canceled)

15. A method for treating a condition in a subject, wherein said condition is characterized by omics-discoverable features, the method comprising:
a. obtaining a biological sample from at least one subject, wherein said biological sample comprises cell-free nucleic acids;
b. sequencing said cell-free nucleic acids;
c. mapping the sequencing results to the reference human genome;
d. identifying unmapped non-linear reads;
e. mapping the unmapped non-linear reads to the pre-computed sequence data set indicative of said condition;
f. identifying at least one sequence associated with said condition;
g. applying a pre-computed treatment model to identify therapeutic means suitable for treating the condition;
h. identifying the therapeutic means for treating the condition based on the pre-computed treatment model; and
i. providing the subject with the therapeutic means to thereby effectively treat the condition in the subject.

16. The method of claim 15, further comprising the step of isolating circulating cell-free nucleic acids from the biological sample.

17. The method of claim 15, wherein the biological sample is selected from the group consisting of blood, serum, plasma, urine, saliva, amniotic fluid, feces, synovial fluid, peritoneal fluid, tissue biopsy, pleural fluid, lymphatic fluid, mucus, and cerebrospinal fluid (CSF).

18. The method of claim 17, wherein the biological sample is a liquid biological sample.

19. The method of claim 15, wherein the circulating cell-free nucleic acids is selected from the group consisting of circulating cell-free RNA, circulating cell-free nucleic acid complexes, circulating cell-free DNA, circulating cell-free microRNA.

20. The method of claim 15, wherein the condition characterized by omics-discoverable features is an autoimmune disorder.

21. The method of claim 20, wherein the autoimmune disorder is selected from the group consisting of systemic lupus erythematosus (SLE), rheumatoid arthritis (RA), scleroderma, primary Sjogren's syndrome, dermatomyositis, systemic vasculitides, Hashimoto's thyroiditis, Graves' disease, multiple sclerosis (MS), type 1 diabetes mellitus (T1DM), anti-phospholipid (aPL) syndrome, pemphigus vulgaris, auto-immune hemolytic anemia, idiopathic thrombocytopenic purpura, myasthenia gravis, Chronic inflammatory demyelinating polyneuropathy, Guillain-Barre syndrome, psoriasis, Crohn's disease, urticaria, psoriasis, psoriatic arthritis, osteoarthritis, asthma, allergy, and Inflammatory Bowel disease (IBD).

22. The method of claim 21, wherein the autoimmune disorder is systemic lupus erythematosus (SLE).

23. The method of claim 21, wherein the autoimmune disorder is rheumatoid arthritis (RA).

24. The method of claim 15, wherein the at least one sequence associated with said condition is selected from the group consisting of sequences having at least 75% sequence identity to the nucleotide sequence set forth as SEQ ID NO:1-17.

25. The method of claim 15, wherein the at least one sequence associated with said condition is selected from the group consisting of sequences having at least 75% sequence identity to the nucleotide sequence set forth as SEQ ID NO: 18-38.

26. The method of claim 15, wherein the at least one sequence associated with said condition is selected from the group consisting of the nucleotide sequence set forth as SEQ ID NO:1-38.

27. The method of claim 15, wherein omics-discoverable features are selected from the group consisting of genomics-discoverable features, proteomics-discoverable features, metagenomics-discoverable features, methylomics-discoverable features, epigenomics-discoverable features, hypoxia-discoverable features, microbiome-discovered features, and metabolomics-discoverable features.

28. The method of claim 16, wherein omics-discoverable features are selected from chimeras, chimeric RNAs, gene-gene fusions, sense-antisense (SAS) chimeras, exon-intron fusions, exon-exon fusions, intron-exon fusions, genomic integrations, aberrations, pathogen integrations and inversions.

29. The method of claim 16, wherein the therapeutic means are selected from the group consisting of an investigational drug, an approved drug, a food supplement, phototherapy, radiation therapy, surgical intervention, non-invasive image-guided procedure, multi-step treatment protocol, or any combination thereof.

30-42. (canceled)

43. An isolated nucleotide sequence having at least 75% sequence identity to the nucleotide sequence selected from the group consisting of SEQ ID NO: 1-17.

44. An isolated nucleotide sequence having at least 75% sequence identity to the nucleotide sequence selected from the group consisting of SEQ ID NO:18-38.

45. An isolated nucleotide sequence selected from the group consisting of nucleotide sequences set forth as SEQ ID NO: 1-38.

46. (canceled)

47. The method of claim 15, wherein the subject is a human subject.

48. (canceled)


SEQUENCE LISTING (38 sequences)

SEQ ID NO: 1 - DNA, 10772 bp, Artificial
  Synthetic
  misc_feature: Microbiome-derived genomic integration
  misc_feature: comp1079_c0
taagctcaggaaatgcagcagcaagataatcacgagtatcctttcctttatcagcggcagacttgccaccaagtccaaccaaatcaagcaacttatcagaaacggcagaagtgccagcctgcaacgtaccttcaagaagtcctttaccagctttagccatagcaccagaaacaaaactagggacggcctcatcagggttaggaacattagagccttgaatggcagatttaataccagcatcacccatgcctacagtattgttatcggtagcaagcacatcaccttgaatgccaccggaggcggctttttgaccgcctccaaacaatttagacatggcgccaccagcaagagcagaagcaataccgccagcaatagcaccaaacataaatcacctcacttaagtggctggagacaaataatctctttaataacctgattcagcgaaaccaatccgcggcatttagtagcggtaaagttagaccaaaccatgaaaccaacataaacattattgcccggcgtacggggaaggacgtcaatagtcacacagtccttgacggtataataaccaccatcatggcgaccatccaaaggataaacatcataggcagtcgggagggtagtcggaaccgaagaagactcaaagcgaaccaaacaggcaaaaaatttagggtcggcatcaaaagcaatatcagcaccaacagaaacaacctgattagcggcgttgacagatgtatccatctgaatgcaatgaagaaaaccaccattaccagcattaaccgtcaaactatcaaaatataacgttgacgatgtagctttaggtgtctgtaaaacaggtgccgaagaagctggagtaacagaagtgagaaccagcttatcagaaaaaaagtttgaattatggcgagaaataaaagtctgaaacatgattaaactcctaagcagaaaacctaccgcgcttcgcttggtcaacccctcagcggcaaaaattaaaatttttaccgcttcggcgttataacctcacactcaatcttttatcacgaagtcatgattgaatcgcgagtggtcggcagattgcgataaacggtcacattaaatttaacctgactattccactgcaacaactgaacggactggaaacactggtcataatcatggtggcgaataagtacgcgttcttgcaaatcaccagaaggcggttcctgaatgaatgggaagccttcaagaaggtgataagcaggagaaacatacgaaggcgcataacgataccactgaccctcagcaatcttaaacttcttagacgaatcaccagaacggaaaacatccttcatagaaatttcacgcggcggcaagttgccatacaaaacagggtcgccagcaatatcggtataagtcaaagcacctttagcgttaaggtactgaatctctttagtcgcagtaggcggaaaacgaacaagcgcaagagtaaacatagtgccatgctcaggaacaaagaaacgcggcacagaatgtttataggtctgttgaacacgaccagaaaactggcctaacgacgtttggtcagttccatcaacatcatagccagatgcccagagattagagcgcatgacaagtaaaggacggttgtcagcgtcataagaggttttacctccaaatgaagaaataacatcatggtaacgctgcatgaagtaatcacgttcttggtcagtatgcaaattagcataagcagcttgcagacccataatgtcaatagatgtggtagaagtcgtcatttggcgagaaagctcagtctcaggaggaagcggagcagtccaaatgtttttgagatggcagcaacggaaaccataacgagcatcatcttgattaagctcattagggttagcctcggtacggtcaggcatccacggcgctttaaaatagttgttatagatattcaaataaccctgaaacaaatgcttagggattttattggtatcagggttaatcgtgccaagaaaagcggcatggtcaatataaccagtagtgttaacagtcgggagaggagtggcattaacaccatccttcatgaacttaatccactgttcaccataaacgtgacgatgagggacataaaaagtaaaaatgtctacagtagagtcaatagcaaggccacgacgcaatggagaaagacggagagcgccaacggcgtccatctcgaaggagtcgccagcgataaccggagtagttgaaatggtaataagacgaccaatctgaccagcaaggaagccaagatgggaaaggtcatgcggcatacgctcggcgccagtttgaatattagacataatttatcctcaagtaaggggccgaagcccctgcaattaaaattgttgaccacctacataccaaagacgagcgcctttacgcttgcctttagtacctcgcaacggctgcggacgaccagggcgagcgccagaacgttttttacctttagacattacatcactccttctgcacgtaatttttgacgcacgttttcttctgcgtcagtaagaacgtcagtgtttcctgcgcgtacacgcaaggtaaacgcgaacaattcagcggctttaaccggacgctcgacgccattaataatgttttccgtaaattcagcgccttccatgatgagacaggccgtttgaatgttgacgggatgaacataataagcaatgacggcagcaataaactcaacaggagcaggaaagcgagggtatcctacaaagtccagcgtaccataaacgcaagcctcaacgcagcgacgagcacgagagcggtcagtagcaatccaaactttgttactcgtcagaaaatcgaaatcatcttcggttaaatccaaaacggcagaagcctgaatgagcttaatagaggccaaagcggtctggaaacgtacggattgttcagtaacttgactcatgatttcttacctattagtggttgaacagcatcggactcagatagtaatccacgctcttttaaaatgtcaacaagagaatctctaccatgaacaaaatgtgactcatatctaaaccagtccttgacgaacgtgccaagcatattaagccacttctcctcatccaacgcgtcagtttttgacagaatcgttagttgatggcgaaaggtcgcaaagtaagagcttctcgagctgcgcaaggataggtcgaattttctcattttccgccagcagtccacttcgatttaattcgtaaacaagcagtagtaattcctgctttatcaagataatttttcgactcatcagaaatatccgaaagtgttaacttctgcgtcatggaagcgataaaactctgcaggttggatacgccaatcatttttatcgaagcgcgcataaatttgagcagatttgtcgtcacaggttgcgccgccaaaacgtcggctacagtaacttttcccagcctcaatctcatctctctttttgcgttctgcttcaatatctggttgaacggcgtcgcgtcgtaacccagcttggtaagttggattaagcactccgtggacagatttgtcattgtgagcattttcatcccgaagttgcggctcattctgattctgaacagcttcttgggaagtagcgacagcttggtttttagtgagttgttccattctttagctcctagacctttagcagcaaggtccatatctgactttttgttaacgtatttagccacatagaaaccaacagccatataactggtagctttaagcggctcacctttagcatcaacaggccacaaccaaccagaacgtgaaaaagcgtcctgcgtgtagcgaactgcgatgggcatactgtaaccataaggccacgtattttgcaagctatttaactggcggcgattgcgtacccgacgaccaaaattagggtcaacgctacctgtaggaagtgtccgcataaagtgcaccgcatggaaatgaagacggccattagctgtaccatactcaggcacacaaaaatactgatagcagtcggcgtgtgaatcattagccttgcgaccctcggcagcaagaaccatacgaccaatatcacgaaaatagtcacgcaaagcattgggattatcataaaacgcctctaatcggtcgtcagccaacgtgagagtgtcaaaaacgataaaccaaccatcagcatgagcctgtcgcattgcattcatcaaacgctgaatagcaaagcctctacgcgatttcatagtggaggcctccagcaatcttgaacactcatccttaatacctttctttttggggtaattatactcatcgcgaatatccttaagagggcgttcagcagccagcttgcggcaaaactgcgtaaccgtcttctcgttctctaaaaaccatttttcgtccccttcggggcggtggtctatagtgttattaatatcaagttgggggagcacattgtagcattgtgccaattcatccattaacttctcagtaacagatacaaactcatcacgaacgtcagaagcagccttatggccgtcaacatacatatcaccattatcgaactcaacgccctgcatacgaaaagacagaatctcttccaagagcttgatgcggttatccatctgcttatggaagccaagcattggggattgagaaagagtagaaatgccacaagcctcaatagcaggtttaagagcctcgatacgctcaaagtcaaaataatcagcgtgacattcagaagggtaataagaacgaaccataaaaaagcctccaagatttggaggcatgaaaacatacaattgggagggtgtcaatcctgacggttatttcctagacaaattagagccaataccatcagctttaccgtctttccagaaattgttccaagtatcggcaacagctttatcaataccatgaaaaatatcaaccacaccagaagcagcatcagtgacgacattagaaatatcctttgcagtagcgccaatatgagaagagccataccgctgattctgcgtttgctgatgaactaagtcaacctcagcactaaccttgcgagtcatttctttgatttggtcattggtaaaatactgaccagccgtttgagcttgagtaagcatttggcgcataatctcggaaacctgctgttgcttggaaagattggtgttttccataatagacgcaacgcgagcagtagactccttctgttgataagcaagcatctcattttgtgcatatacctggtctttcgtattctggcgtgaagtcgccgactgaatgccagcaatctctttttgagtctcattttgcatctcggcaatctctttctgattgtccagttgcattttagtaagctctttttgattctcaaatccggcgtcaaccataccagcagaggaagcatcagcaccagcacgctcccaagcattaagctcaggaaatgcagcagcaagataatcacgagtatcctttcctttatcagcggcagacttgccaccaagtccaaccaaatcaagcaacttatcagaaacggcagaagtgccagcctgcaacgtaccttcaagaagtcctttaccagctttagccatagcaccagaaacaaaactagggacggcctcatcagggttaggaacattagagccttgaatggcagatttaataccagcatcacccatgcctacagtattgttatcggtagcaagcacatcaccttgaatgccaccggaggcggctttttgaccgcctccaaacaatttagacatggcgccaccagcaagagcagaagcaataccgccagcaatagcaccaaacataaatcacctcacttaagtggctggagacaaataatctctttaataacctgattcagcgaaaccaatccgcggcatttagtagcggtaaagttagaccaaaccatgaaaccaacataaacattattgcccggcgtacggggaaggacgtcaatagtcacacagtccttgacggtataataaccaccatcatggcgaccatccaaaggataaacatcataggcagtcgggagggtagtcggaaccgaagaagactcaaagcgaaccaaacaggcaaaaaatttagggtcggcatcaaaagcaatatcagcaccaacagaaacaacctgattagcggcgttgacagatgtatccatctgaatgcaatgaagaaaaccaccattaccagcattaaccgtcaaactatcaaaatataacgttgacgatgtagctttaggtgtctgtaaaacaggtgccgaagaagctggagtaacagaagtgagaaccagcttatcagaaaaaaagtttgaattatggcgagaaataaaagtctgaaacatgattaaactcctaagcagaaaacctaccgcgcttcgcttggtcaacccctcagcggcaaaaattaaaatttttaccgcttcggcgttataacctcacactcaatcttttatcacgaagtcatgattgaatcgcgagtggtcggcagattgcgataaacggtcacattaaatttaacctgactattccactgcaacaactgaacggactggaaacactggtcataatcatggtggcgaataagtacgcgttcttgcaaatcaccagaaggcggttcctgaatgaatgggaagccttcaagaaggtgataagcaggagaaacatacgaaggcgcataacgataccactgaccctcagcaatcttaaacttcttagacgaatcaccagaacggaaaacatccttcatagaaatttcacgcggcggcaagttgccatacaaaacagggtcgccagcaatatcggtataagtcaaagcacctttagcgttaaggtactgaatctctttagtcgcagtaggcggaaaacgaacaagcgcaagagtaaacatagtgccatgctcaggaacaaagaaacgcggcacagaatgtttataggtctgttgaacacgaccagaaaactggcctaacgacgtttggtcagttccatcaacatcatagccagatgcccagagattagagcgcatgacaagtaaaggacggttgtcagcgtcataagaggttttacctccaaatgaagaaataacatcatggtaacgctgcatgaagtaatcacgttcttggtcagtatgcaaattagcataagcagcttgcagacccataatgtcaatagatgtggtagaagtcgtcatttggcgagaaagctcagtctcaggaggaagcggagcagtccaaatgtttttgagatggcagcaacggaaaccataacgagcatcatcttgattaagctcattagggttagcctcggtacggtcaggcatccacggcgctttaaaatagttgttatagatattcaaataaccctgaaacaaatgcttagggattttattggtatcagggttaatcgtgccaagaaaagcggcatggtcaatataaccagtagtgttaacagtcgggagaggagtggcattaacaccatccttcatgaacttaatccactgttcaccataaacgtgacgatgagggacataaaaagtaaaaatgtctacagtagagtcaatagcaaggccacgacgcaatggagaaagacggagagcgccaacggcgtccatctcgaaggagtcgccagcgataaccggagtagttgaaatggtaataagacgaccaatctgaccagcaaggaagccaagatgggaaaggtcatgcggcatacgctcggcgccagtttgaatattagacataatttatcctcaagtaaggggccgaagcccctgcaattaaaattgttgaccacctacataccaaagacgagcgcctttacgcttgcctttagtacctcgcaacggctgcggacgaccagggcgagcgccagaacgttttttacctttagacattacatcactccttctgcacgtaatttttgacgcacgttttcttctgcgtcagtaagaacgtcagtgtttcctgcgcgtacacgcaaggtaaacgcgaacaattcagcggctttaaccggacgctcgacgccattaataatgttttccgtaaattcagcgccttccatgatgagacaggccgtttgaatgttgacgggatgaacataataagcaatgacggcagcaataaactcaacaggagcaggaaagcgagggtatcctacaaagtccagcgtaccataaacgcaagcctcaacgcagcgacgagcacgagagcggtcagtagcaatccaaactttgttactcgtcagaaaatcgaaatcatcttcggttaaatccaaaacggcagaagcctgaatgagcttaatagaggccaaagcggtctggaaacgtacggattgttcagtaacttgactcatgatttcttacctattagtggttgaacagcatcggactcagatagtaatccacgctcttttaaaatgtcaacaagagaatctctaccatgaacaaaatgtgactcatatctaaaccagtccttgacgaacgtgccaagcatattaagccacttctcctcatccaacgcgtcagtttttgacagaatcgttagttgatggcgaaaggtcgcaaagtaagagcttctcgagctgcgcaaggataggtcgaattttctcattttccgccagcagtccacttcgatttaattcgtaaacaagcagtagtaattcctgctttatcaagataatttttcgactcatcagaaatatccgaaagtgttaacttctgcgtcatggaagcgataaaactctgcaggttggatacgccaatcatttttatcgaagcgcgcataaatttgagcagatttgtcgtcacaggttgcgccgccaaaacgtcggctacagtaacttttcccagcctcaatctcatctctctttttgcgttctgcttcaatatctggttgaacggcgtcgcgtcgtaacccagcttggtaagttggattaagcactccgtggacagatttgtcattgtgagcattttcatcccgaagttgcggctcattctgattctgaacagcttcttgggaagtagcgacagcttggtttttagtgagttgttccattctttagctcctagacctttagcagcaaggtccatatctgactttttgttaacgtatttagccacatagaaaccaacagccatataactggtagctttaagcggctcacctttagcatcaacaggccacaaccaaccagaacgtgaaaaagcgtcctgcgtgtagcgaactgcgatgggcatactgtaaccataaggccacgtattttgcaagctatttaactggcggcgattgcgtacccgacgaccaaaattagggtcaacgctacctgtaggaagtgtccgcataaagtgcaccgcatggaaatgaagacggccattagctgtaccatactcaggcacacaaaaatactgatagcagtcggcgtgtgaatcattagccttgcgaccctcggcagcaagaaccatacgaccaatatcacgaaaatagtcacgcaaagcattgggattatcataaaacgcctctaatcggtcgtcagccaacgtgagagtgtcaaaaacgataaaccaaccatcagcatgagcctgtcgcattgcattcatcaaacgctgaatagcaaagcctctacgcgatttcatagtggaggcctccagcaatcttgaacactcatccttaatacctttctttttggggtaattatactcatcgcgaatatccttaagagggcgttcagcagccagcttgcggcaaaactgcgtaaccgtcttctcgttctctaaaaaccatttttcgtccccttcggggcggtggtctatagtgttattaatatcaagttgggggagcacattgtagcattgtgccaattcatccattaacttctcagtaacagatacaaactcatcacgaacgtcagaagcagccttatggccgtcaacatacatatcaccattatcgaactcaacgccctgcatacgaaaagacagaatctcttccaagagcttgatgcggttatccatctgcttatggaagccaagcattggggattgagaaagagtagaaatgccacaagcctcaatagcaggtttaagagcctcgatacgctcaaagtcaaaataatcagcgtgacattcagaagggtaataagaacgaaccataaaaaagcctccaagatttggaggcatgaaaacatacaattgggagggtgtcaatcctgacggttatttcctagacaaattagagccaataccatcagctttaccgtctttccagaaattgttccaagtatcggcaacagctttatcaataccatgaaaaatatcaaccacaccagaagcagcatcagtgacgacattagaaatatcctttgcagtagcgccaatatgagaagagccataccgctgattctgcgtttgctgatgaactaagtcaacctcagcactaaccttgcgagtcatttctttgatttggtcattggtaaaatactgaccagccgtttgagcttgagtaagcatttggcgcataatctcggaaacctgctgttgcttggaaagattggtgttttccataatagacgcaacgcgagcagtagactccttctgttgataagcaagcatctcattttgtgcatatacctggtctttcgtattctggcgtgaagtcgccgactgaatgccagcaatctctttttgagtctcattttgcatctcggcaatctctttctgattgtccagttgcattttagtaagctctttttgattctcaaatccggcgtcaaccataccagcagaggaagcatcagcaccagcacgctcccaagcat

SEQ ID NO: 2 - DNA, 1693 bp, Artificial
  Synthetic
  misc_feature: Microbiome-derived genomic integration
  misc_feature: comp11383_c1
tgtcgtgtgggtggggtgtccggttttccatgccccacctcccacctaccgccccgcgcccatgccctgccgccagcccggccgcaacccatcttcacgtccggggtgctgggcccctacttgcaaggccttggccttgtcggggcctcaccgcgctttacttacctggttcatcctgccagtaacatgtttgtctgagagattaagccatgcgtatctaagtatgcatggccggtacagtgaaactgcgaatggctcattaaatcagttatggttcctttggtcgctcgctcctctcctacttggataactgtggtaattctagagctaatacatgccgacgggcgctgacccccttcgcgggggggatgcgtgcatttatcagatcaaaaccaacccggtcagcccctcttcagcccaggccgggggaggggttcgggtgccactggctttggtgactctagatagccttgggttaatcgcacgccccccgtggcagggacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccacggtaattccagctccaataacgtatattaaagttgctgcacttaaaaagctcgtagttggatcttggggacgggtggtgtcacctctatgctcttagctgagtgtcctgtggggcccgaagtgtttgaaaaaactagtgttcaaagcaggcccgagccacctggattctgaagctaggaataatggaagaggaccgtggttctattttgtgggtttttgaactgaggccatgattaagagggacggccggggggctttcatattgcacctctagaggtgaaattcttggaccggcgcaagacggaccagagcgaaagcatttgccaagaatgttttcattaatcaagaatgaaagtcggaggtttgaagatgatcagataccgtcataattcccaccataaacgatgctgactggtgatgtggtggcattattcccatgacctgccaggcagcttccgggaaaccaaagtctttgggttccggtgggagtatggttgcaaagctgaaacaaaggaattaacagaagggcaccaccaggagtggagcctgtggcttaatttgactcaacacgggaaacctcacccggacagggacaggattgacagctctttctcgattccgtgagtggtggtgcatggctgttcttggtggagcgatttgtctggctaattccgataataaacgagactctggcatgctaactcattatgcgaccccagagtggtcagcgtcctcaaacttcttagagggacaagtggcattcagccacccgagattgagcagtaacaggtctgtgatgcccttagatgtccggggctgcatgtgtgttacactggctcagcgtgtgcctaccctatgctggcaggtgcgggtaacccattgaacctcatttgtgatggggat

SEQ ID NO: 3 - DNA, 583 bp, Artificial
  Synthetic
  misc_feature: Microbiome-derived genomic integration
  misc_feature: comp10763_c0
gtatatcttttcagtaacaccccactctactggtaccaatttactatattagttcattttcacactgctgaaaaagacatacccaaaagtgggaacaaaaagaggtttaattggacttacagttccacatggctggggaggcctcacaatcatggcagaaggcaaggaggagcaagtcacatctcacatggatggcggcaggcaaagagggagaacttgtgaaggagaactcctctttataaaaccatcagatctcatgagacttattccctaccacgaaaacagcacaagaaagacctgcccccaagattcaattacctcccactgggcccctcctatgacacgtgggaattgtgagagttacaattcaaggtgagatttgggtggggacacagccaaattttgtgtccccacccatgtgatggtgaggatggtgatggtgacaatggtgatgatgatgatgacaatgatgatggtgatgattatgatgatgatggtggtggtgggtgatgatgatgttgacaatgatgataatggtgattatgatgacaatgatgacgatgatggtgattatgatgatgat

SEQ ID NO: 4 - DNA, 428 bp, Artificial
  Synthetic
  misc_feature: Microbiome-derived genomic integration
  misc_feature: comp33944_c0
gcttgggcaacagagcgagactccatataaaaaaaaaaaaaaaggagttcccctgcacaagctctctctctttgcctgctgccatccatctaagatgtgacttgctaccccttcatcttctgccataattgtaagtttcctgaggcctcccagtcatgctttctgtacagcctgcagaactgtgagtcaattaaacttctttcctttataaattacccagtttcaggtatgtctttattagcagcgtgagaacagactaatacagtggactttcattacatttcttatagacaggtctgctggtaataattatcttcagcttttatgtgtcataaaagacctttatttttctttctctctctttctctctttttctttctctttctttctttcttcctttcttcctttctttctttctttcttttttt

SEQ ID NO: 5 - DNA, 429 bp, Artificial
  Synthetic
  misc_feature: Microbiome-derived genomic integration
  misc_feature: comp9841_c0
attcatctgtgtctttctaaagggagtggaagagctggggtaaatttgatggagtctagaatactgaaggcgtaaatggcgaaatacatgaataaggcttcccaagggtggcttttcccctgctccttccttccttctccctccccttttcctcctctggctgcttagcacccacatcccttggctatgttggggaatctcttcttctccccaccttgtgggtcttggtggaaggcagacctgcctttcaggatagaagttggatttcaggctgggtgtggtggctcacacctgtaatcccagcactttgggagaccaaggcaggtggatcacttgaggtcaggagttcaaaaccagcctggccaacatggtgaaatcccatctctactaaaaatacaaaattagccaggcatggtggcacatgcctgt

SEQ ID NO: 6 - DNA, 519 bp, Artificial
  Synthetic
  misc_feature: Microbiome-derived genomic integration
  misc_feature: comp86121_c0
gtgactggagttcagacgtgtgcgcttccatctccactaccagtccactgactcaaatgttaatctcctttggcaacaccctcacagacacacccaggaacaatactttgcattcttcaatccaatcaagttaacactcaatattaaccatcacagggatgacagtgatgatgacgacagtgatgatgatgatgatgttgttgataatgatggcaatggtgatagtggtgatgatggtgatgataatgttgatagtgatggtgatggtgatggctatgatgatggtagtgatggtgatggtgatgttggtagtgatggtgatcatggtgatggggatggggatgaggatggggatgatgatgatgacaatgaggtgatgatgatgttgatagtgatggtgatggtgatggctatgatgatggtagtgatggtgatggtgatgttggtagtgatggtgatcatggtgatggggatggggatgaggatggggatgatgatgatgacaatgaggtgatgatg

SEQ ID NO: 7 - DNA, 411 bp, Artificial
  Synthetic
  misc_feature: Microbiome-derived genomic integration
  misc_feature: comp5051_c0
tgccttgcttccccttcaccttctaccatgattgtaagtttcctgaggtctccccagccatgtggaactgtgagtcaattaaacctctttcctttataaattacccagtcttgggtatgtctttattagcagcatgagaacagactaatacagtaaattggtaccagtagagtggggcattgctgaaaagatacccaaaaatgtggaagcagctttggaactgggtaatgggcagaggttagaaggatgtggagggctcacccaacagtcaggaaagagatgtaacacgaagctgatgatacagttaaatgttagtttcattgctaaagaaaccttgctagggctttttaaaaacctctattcagaactgtggctcttagtgagaaaacaaaaatattaacattgctcaca

SEQ ID NO: 8 - DNA, 431 bp, Artificial
  Synthetic
  misc_feature: Microbiome-derived genomic integration
  misc_feature: comp725_c0
atgtgtacccactaaccctccccattttcccaaccctgcccccccatctgaccccacctactacccttcacagcctctggtaaccattcttctactctctatctccatgagttcaattgttttaatttttagctcccacaaataagtgaaaacatgtgaagttgccttcctgtgctggcttatttcacttaacataattatctccatttgcatccacattggtgcaaataacagaatctcattattttttatagctgaatagtattctactgcatgtttgtgtgtgtgtatgtgtgtgtgtgcacatgtgtgtgtgtggatgtgcctgcatccaggtgtgtgggtgtgtgtgtgtgtgggtgtgtgtttgtgtgtgtatataaacaaatttatatatatatatttatataatttttttttttttttttttg

SEQ ID NO: 9 - DNA, 362 bp, Artificial
  Synthetic
  misc_feature: Microbiome-derived genomic integration
  misc_feature: comp141778_c0
tcgcccaggctggagtgcagtgaagccatgcttcctgtacagcctgaggaactccaagtatataaaaatgtaaaacagttaaacttacacaaacatagagctgaatcgtggttaccaagggctgagggggaagggtaactgggggggtattggtcgcagggtataagttttagttatgcaatgagtaagttctaaatatcgaatctacagatcaaagcctgtagttaatacggaattgcatacttaaaaatttgttaagaggatggtattgtttcgatttgtttacttgcccaaatctcatgtcaaattgtaatccccagtgttggaagtggggcctggtgggaggtgtttgggtcatgggg

SEQ ID NO: 10 - DNA, 367 bp, Artificial
  Synthetic
  misc_feature: Microbiome-derived genomic integration
  misc_feature: comp262756_c0
gctcagcgttctcctcccaccacccaccatgctggactcttcccaggtcagcacttaagcgatgcctttgcccaggtgaaccccctcaagaaggtgccagccttgaaggacggggacttcaccttgacggagaggtaactgggaccctaggactgctgccaggcctgctggaaccatcctgttctaaccctctatttcatagacaaggaaactaaagtcttcagaggcagagagtctctgtgccccgcatcctgcagagagtcagtactggaccccaggcccttgccttcctgctattccagttcaccctgatgattagaaaagcaaatatctactttacttccacaccagtgctttggttgctggt

SEQ ID NO: 11 - DNA, 422 bp, Artificial
  Synthetic
  misc_feature: Microbiome-derived genomic integration
  misc_feature: comp186508_c0
ggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgac

SEQ ID NO: 12 - DNA, 569 bp, Artificial
  Synthetic
  misc_feature: Microbiome-derived genomic integration
  misc_feature: comp231082_c0
gagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtctagctcgaccaggatgggcaccaccccggtgaacagctcctcgcccttgctcaccatggtggcgaccggtggatcccgggcccgcggta

SEQ ID NO: 13 - DNA, 336 bp, Artificial
  Synthetic
  misc_feature: Microbiome-derived genomic integration
  misc_feature: comp89065_c0
gaagcgcatgaactccttgatgatggccatgttattctcctcgcccttgctcaccatggtggcgaccggtggatcccgggcccgcggtaccgtcgactgcagaattcgaagcttgagctcgagatctgagtccggtagcgctagcggatctgacggttcactaaaccagctctgcttatatagacctcccaccgtacacgcctaccgcccatttgtgtcaatggggcggagttgttacgacattttggaaagtcccgttgattttggtgccaaaacaaactcccattgacgtcaatggggtggagacttggaaatccccgtgagtcaaaccgctat

SEQ ID NO: 14 - DNA, 335 bp, Artificial
  Synthetic
  misc_feature: Microbiome-derived genomic integration
  misc_feature: comp89065_c0
gcggttggatggcaccggggatatggatcggctcatcgtcgcaggtcaagaggtcaggctgcctggcgacggatccttcagccatggtggcggcagatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaa

SEQ ID NO: 15 - DNA, 309 bp, Artificial
  Synthetic
  misc_feature: Microbiome-derived genomic integration
  misc_feature: comp153629_c0
aaaaagcaggctccatggactgacaagagtatagtggttaacttcacctccagactcttcgctgatgaactggccgcccttcagtcaaaaatagggagcgtactgccgctcggagattgccaccgtttacaaaatatacaggcattgggcctggggtgcgtatgctcacgtgagacatctccggactacatccaaattatgcagtatctatccaagtgcacactcgctgtcctggaggaggttcgcccggacagcctgcgcctaacgcggatggatccctctgacaaccttcagataaaaaacgtatat

SEQ ID NO: 16 - DNA, 320 bp, Artificial
  Synthetic
  misc_feature: Microbiome-derived genomic integration
  misc_feature: comp429_c0
actttatcacattatttgcatgttacctcctataaacttagtgcggacaagttttaatccagaattgaccttttgacttaaagcagggggactttgtatagaaggtttgggggctgtggggaaggagaactgttggaaaggcagaggatggttactgtcatgctgttctcatgatagtgagttctcatgagatcttgttgtttaaaagtgtgtggcacctgcccccccccctctcccctacgccagccaaggaagaactacttgcttccccatcaaattcccctaattttgtaatgttgttgacgccccccccacagccg

SEQ ID NO: 17 - DNA, 338 bp, Artificial
  Synthetic
  misc_feature: Microbiome-derived genomic integration
  misc_feature: comp139079_c0
aggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccatagtcccgcccctaactccgcccatcccgcccctaactccgcccagttccgcccattctccggcccctggctgactaattttttttagctagga

SEQ ID NO: 18 - DNA, 650 bp, Artificial
  Synthetic
  misc_feature: Predicted druggable chimera
aggccagccacacatataaccctttccctactttactaatgtatcccttatgtggtaccagcaatggaggacaggcagacttaccccctgccatctagagagaatgttgttattacccgtaaaacttgaccacccccatatcccactcctttttgtaaaaacaaatgcttaaacctgtgagcctgccgttcctttctatgtgttaatcagtttccttccatttgagctgtgtgggagggaagggcattgaaattgtaggttgtaatcttgtgccaaccaataaaaaccagtatttcaatgcccttccctcccacacagctcaaatggaaggaaactgattaacacatagaaaggaacggcaggctcacaggtttaagcatttgtttttacaaaaaggagtgggatatgggggtggtcaagttttacgggtaataacaacattctctctagatggcagggggtaagtctgcctgtcctccattgctggtaccacataagggatacattagtaaagtagggaaagggttatatgtgtggctggcctgttctttagagaggaaggtgggatggtatctgctgtgtacctgtctcagaccaaacctggggctggaccagtgccccttctcagccctcctccacagcctgactgt

SEQ ID NO: 19 - DNA, 490 bp, Artificial
  Synthetic
  misc_feature: Predicted druggable chimera
  misc_feature: SULF2/NCOA3
gatgaaaaggtaacataattaaacatacacacacacacacacacacacatacacacacacacagcccatgaattgctattatttcggactgcttaaaatccaaaagactctcttcaaattgctcgtttttttaaattcctgatttttttttaagtttatttccccatatctattctttccccccaaattaacaaagtgcttagttctggtagaggaatttttaaaaaattaaaatgcctacatctaaacaaataatctctttaaaaagcatttagattaagagattgaaaagagcgaggcatctaaggctggggggtggggaaggaaaagctgtggagcactacccctcccccaggccacaaaagtcaagggatgtctcttccactggtagcctcaccacacagcttactgaaatcaacaatgttaaatatttagcttcacaaactctgtaaaaatgagttgttgcgacctacaaaaaaaggtatataca

SEQ ID NO: 20 - DNA, 4876 bp, Artificial
  Synthetic
  misc_feature: Predicted druggable chimera
ggcacgagggcgcagacggggaagcggagccaacatgccagtggcccggagctgggtttgtcgcaaaacttatgtgaccccgcggagacccttcgagaaatctcgtctcgaccaagagctgaagctgatcggcgagtatgggctccggaacaaacgtgaggtctggagggtcaaatttaccctggccaagatccgcaaggccgcccgggaactgctgacgcttgatgagaaggacccacggcgtctgttcgaaggcaacgccctgctgcggcggctggtccgcattggggtgctggatgagggcaagatgaagctggattacatcctgggcctgaagatagaggatttcttagagagacgcctgcagacccaggtcttcaagctgggcttggccaagtccatccaccacgctcgcgtgctgatccgccagcgccatatcagggtccgcaagcaggtggtgaacatcccgtccttcattgtccgcctggattcccagaagcacatcgacttctctctgcgctctccctacgggggtggccgcccgggccgcgtgaagaggaagaatgccaagaagggccagggtggggctggggctggagacgacgaggaggaggattaagtccacctgtccctcctgggctgctggattgtctcgttttcctgccaaataaacaggatcagcgctttccaaaaaaaaaaaaaaaaaaaaaaaaaaaaaaaaaaaaaaaaaaaaaaaaaaactcgagttttttttttttttttctaagtaagaaatttatttgatcaaatggcaatatatgaagatattttaaactataaggctctatccagacaaaaagcatctttttataaagtaagtgttatcaatttaaatggcacctaaaatcaaagaatgagtttcctataaatgtctgtataaagctttcataaaatcctcatgtattttaaaactaggttgcaagtataacagaaaaacctataaatagtacttaaaagcaggtttcttaaaatttatgacacctacagtgtgtcactatagacagagtattcggggggctctgtgacaatcaaacatggctgccatgagtaagggagcgtgtcccctgatgagcagaaccgaagcagtgattatgcctcagcctcgagctccccgcacagcatcctaccattccaaattcttgggactttgagttgagttgagaattactacaagaggaggttaggaaaccaggattggaaagagggaagggagaggaaaccaaggcatgccaggatcaaagatatctataaagcagctttataaatgaccaagaaaatgggtttctaaaaaacgcaaaatggtgaggccttggggctgcaacgtggcatctgtgaagaggacgacgccacgcccgctgggaagggctgacctgcatgccaggtgcttggcttggagccaccacacgcaacagcatgaaactttctctggagtgccaagtcattacctggaaacactggggacaggtcagtgtgtagcttataccagctttccccaaagatttttccagcaaccaagtgactcagcaaccaagtgactctgtctttggggccgtgatgggagctgacatggaggaatctgcagctccaggttctaagatcaggagagcttcagacaagtgggaaaaccagatataaagagggtgacatacacacactgttagcagcaaaacgcaaaggtgtccaatgattaaaatgaaattgagtgatagggagaaatggagaagattccagagtgtcagggatagtaactgaaaataattatatagcacccattattactaatttaagatatataagcatcactgcggatttatatagtgtctatgctattagtttagtaaatataaatatttatgagggctttcaaaatattttaaatgacatatacattctaatataaatattacactccagattcatacaactgagttgatgttacatttaaagtagtagatttggggttgtatatggagtaacttttgcagaccattttctgaactttgatcattcagctaagtaaaggccaaaaaacacaggaagcatcttattgggaactttggcagcaaacatgtttttatctatgtatttatgcattatacaaagatacacttattcatttgtataaatgaatttaaagtactccataggagatcttacacttaaaacacttttctgtcttcttatctgcttctttgagatcaagtctatatgggaggcttcttccttagaggtgtgtgtttaaaaatagtctcacgtttcccattcaggctaagtggctcttttaagattcctaatcttgaggttacaatcttcacattgagacactgatacacacacatactttaataaaaacagcaaatggtcttatatcacctacttttagcagtgtgcttttaaaacaggtttgttcccagcattacagaaaataatctccaaacacatgcattactttctactcagggaaggatgttacacctaatgactgtatagagtgattttgtaaattagtcttatgcgcgtgtaaactaaatgggctgcccaacttaaccccaatggttaaaggaaagtagtctcggcactatgacatcacagttacacctgtgctctcaggtacagggcgaaagtgagagcgagagcgcacgagagagttcaacctgagagagctttataatttcaagcctaggctgtcattcacctttgcttcatttctccaaaacaagctcctaatttagctacctgaagagactgctgcagcagtacagtgaaagcagcctttttttgaaatagggtctcactctgttgcccaggctggagtgcagtggtgtgatctgtgctcactgcaacctcaacctcccaggttcaagtgaacctcccacctcagccttctgagtagctgggaccacaggtatgtaccaccacacccagctaattttttttgtagagatggggtttagccatgttgcccaggctggtcttgaactcctgggcttaagcaatccacccaccttggccccccaaagtgctgtgattacatgtgtgagccactgtgtccagtcgacagtagcctttttactgtgatgtcaataaaccaacatctgaagatgacaggaactggattcagagtctgtgtaaattttctcaatgagtgaccgagcatgtaaatttaactcaaatgaaattactacaataattgctttaaaaacatatttaggaaaatgaggtgaccctagatgagcttatttatatgtctcctctaacagaaatcacctcatggtaagagttgcttcttttggctcagttcacctgagaacaacttcatttggaacttttcttagccatttaacgtaacagttctggggtacacgggcaaaggggcttgagaaggcccggaggcgaagccgaagagaagcaactgtgccccggagaagagaagctcgcccattccagactgggaaccagctttcagtgaagatggcagggccagaactgttgctcgactccaacatccgcctctgggtggtcttacccatcgttatcatcactttcttcgtaggcatgatccgccactacgtgtccatcctgctgcagagcgacaagaagctcacccaggaacaagtatctgacagtcaagtcctaattcgaagcagagtcctcagggaaaatggaaaatacattcccaaacagtctttcttgacacgaaaatattatttcaacaacccagaggatggatttttcaaaaaaactaaacggaaggtagtgccaccttctcctatgactgatcctactatgttgacagacatgatgaaagggaatgtaacaaatgtcctccctatgattcttattggtggatggatcaacatgacattctcaggctttgtcacaaccaaggtcccatttccactgaccctccgttttaagcctatgttacagcaaggaatcgagctactcacattagatgcatcctgggtgagttctgcatcctggtacttcctcaatgtatttgggcttcggagcatttactctctgattctgggccaagataatgccgctgaccaatcacgaatgatgcaggagcagatgacgggagcagccatggccatgcccgcagacacaaacaaagctttcaagacagagtgggaagctttggagctgacggatcaccagtgggcactagatgatgtcgaagaagagctcatggccaaagacctccacttcgaaggcatgttcaaaaaggaattacagacctctattttttgaagaccgagcagggattagctgtgtcaggaacttggagttgcacttaaccttgtaactttgtttggagctggcacctcttgaaataaaaaggaggatgcacgagctggcaggcatgcagcaaggcttgttcttgtctgggctgggttcccctttatgtttgaaactagaggaaataggagtatgctgggcaacttgttatttttaaaatgttgtaaatgttccttctggtgactctagtaatgaaagtcagagaaagggggaaacctcaggctactgataatatataaaatcttggccaggcgcagtggctcacgcctgtaatcccaggattttgggaggctgaggcgggtgggtcacgagatcaggagttcaagatcagcctggccaagatggtgaaaccccatctctactaaaaatacaaaaaaaaattagccgggcgtggtggtgggcacctgtaatcccagctacttgggaggctgaggcagagaactgcttaaacctaggaggcggaggatgcagtgagctgacatcgtgccactgcgctccagcctgggcgacagagcaaaactccatctgaaaa

SEQ ID NO: 21 - DNA, 2247 bp, Artificial
  Synthetic
  misc_feature: Predicted druggable chimera
tccgtacaaatatgtggactccagtagcaaatatgaatgggaggaggctacagttcggtgttgcagtgctagatgacaaactgtatgtggttggaggaagagatggactgaagactttgaatactgtagagtgctacaaccccaaaacaaaaacttggagtgtgatgccacctatgtccacacatagacatggccttggtgtggctgtactggaaggtcccatgtatgccgtaggaggacatgatggctggagctatctgaacacagtggaaagatgggaccctcaggctcgccagtggaattttgttgccactatgtctacccctaggagtacagtaggtgtggcagtactaagtggaaaactttatgcagttggtggtcgtgatggaagttcttgtctcaaatcagtagaatgttttgatcctcatactaataagtggacactgtgtgcacagatgtcaaaaaggagaggtggcgtaggagtgacgacctggaatggactgctgtatgctataggggggcacgatgctcccgcatccaacttgacttccagactctcagactgtgtggaaagatatgatcccaaaacagacatgtggactgcagtagcatccatgagcatcagcagagatgcagtgggggtctgtttacttggtgataagttatatgctgttggggggtatgatggacaggcataccttaataccgtggaggcttatgatccccagacaaatgagtggacccaggtattttcacatacttttgaggacagcaaagatcacctggtggccatcaagcagaccatctggaggcaaaactccttatctgaggaattcagaagtcattagactgccctattatctaaagccggcatcttgtactaggcttctttaccaaaaatgtatttaataaaacatttccaacctgtgaaaaaaaaaaaaaaaaatttttttttttttgcttcaaagagcttttctcagagcagggatttattttcattacatgcaacatggacaaacactgttctggttttcatgacaatttgaattcaaagtaatatgtttttctaaaattcagtgtatttatttggccatatggatgctccttgtgttcttggtcacatattaaagaaactggcactttggctgcaagaacaaataaaaaatatcataaatccaactggtctttgatttgggtctaggttaataactaaagaaccattcagcaataatggcttgaacatttatatatcctatgaaaccgcaattagttaagaggctgctgattctaataactatgacaccagcaagggagtgaggggagaaatgataactctggatgccaaattcagagcaaagtatctattatctccttctcacttttgcagtatctataaataaagtggtggggggagaattatatgaataatgttaaataaaagtgcatacagaactgagaaatattttcatggaatttgccacttagttcttaaaattcttataaggaaaataaccatttacaacaaaagactagttacactgttgctgtttagaacatgagagcaaaatgagtaacaatcaaattctctggtttaaacttaattatcttaaaacatgttattctgtaagttgacatctatgccttgaaaattcaaggcagaaagtaaaatcatttagaaagccagaaattccatcaatacatctagacagatgtttgcttgtagtttttggtatccaaaaccttttttccacacatcgcacagatgccttttttgtaggcacagccctggcagtaatgagaacctggttggtgcacagaacttttacaaattctacaagtggagaacttattctttccatatggatcaaatcttgctttttttgaagtcaaagctttattttcattcagctttcttccaccactttctgtggtattcctagcaccacctttccatgtatctggagtgataacagtaccaagtttcttttcacatttttcgcacaccatccttccccacgacacctttttcttcccgatctgaacccctgttgactaatcttgcctgggtttgtgtaggtctgcaggaaggaaggctgaaaaagcggacgaagattttgacttaagtgggactttgtgatttaattttttcttttttttaagtggggaggaaggggaagctagatggactaggagagacttgattttggtgctaaagttccccagttcatatgtgacatctttttaaaa

SEQ ID NO: 22 - DNA, 542 bp, Artificial
  Synthetic
  misc_feature: Predicted druggable chimera
  misc_feature: HNRNPA0/HCFC2
ttttaaagaattgaattctttatttgtgatatccataaacgttgctattctctatttctatccagaaaggcaattttcacctattatcacttttgttcttctcttataaacaacaacttgaatgctattgcaggaaagggctacaaatatacatttgttaaccaagcagaatacacagatattttgctttacaacttgcacctaaaataccagtatacgtagctggttcattagttgtcatagcaatttagggctattgcccaagctatgcatagcagtttacattttcaaacctcatatagaaagggctattgtgatatgaactggcaactacattcctgtgaagcccatctcagttacaagcaaatgtgttaacttccaattctgcaaagaattttgatgtatctgtgctgtgcggatagccaagtaggctgaatattccaaaatattttccgaaggtgaggttggaggttccaggaaaggtggataccttcaacattctttgaaaatctgactgcagaaggagctccaggaaaaccagg

SEQ ID NO: 23 - DNA, 1750 bp, Artificial
  Synthetic
  misc_feature: Predicted druggable chimeras
  misc_feature: SULT2A1/SERPINC1
ggtgcacgcctatagtcccagctactcgggaggctgaggtaggagaatcgtttgaactcaggaggcagaggttgcaatgagccaagatcgcgccactgcactccagcctgggtgacagagcgagaccgtctcaaaaagaaagaagtgactagggttcaaagaaccagttttcaggcggattgcctcagatcacactatctccacttgcccagccctgtggaagattagcggccatgtattccaatgtgataggaactgtaacctctggaaaaaggaaggtttatcttttgtccttgctgctcattggcttctgggactgcgtgacctgtcacgggagccctgtggacatctgcacagccaagccgcgggacattcccatgaatcccatgtgcatttaccgctccccggagaagaaggcaactgaggatgagggctcagaacagaagatcccggaggccaccaaccggcgtgtctgggaactgtccaaggccaattcccgctttgctaccactttctatcagcacctggcagattccaagaatgacaatgataacattttcctgtcacccctgagtatctccacggcttttgctatgaccaagctgggtgcctgtaatgacaccctccagcaactgatggaggtatttaagtttgacaccatatctgagaaaacatctgatcagatccacttcttctttgccaaactgaactgccgactctatcgaaaagccaacaaatcctccaagttagtatcagccaatcgcctttttggagacaaatcccttaccttcaatgagacctaccaggacatcagtgagttggtatatggagccaagctccagcccctggacttcaaggaaaatgcagagcaatccagagcggccatcaacaaatgggtgtccaataagaccgaaggccgaatcaccgatgtcattccctcggaagccatcaatgagctcactgttctggtgctggttaacaccatttacttcaagggcctgtggaagtcaaagttcagccctgagaacacaaggaaggaactgttctacaaggctgatggagagtcgtgttcagcatctatgatgtaccaggaaggcaagttccgttatcggcgcgtggctgaaggcacccaggtgcttgagttgcccttcaaaggtgatgacatcaccatggtcctcatcttgcccaagcctgagaagagcctggccaaggtggagaaggaactcaccccagaggtgctgcaagagtggctggatgaattggaggagatgatgctggtggtccacatgccccgcttccgcattgaggacggcttcagtttgaaggagcagctgcaagacatgggccttgtcgatctgttcagccctgaaaagtccaaactcccaggtattgttgcagaaggccgagatgacctctatgtctcagatgcattccataaggcatttcttgaggtaaatgaagaaggcagtgaagcagctgcaagtaccgctgttgtgattgctggccgttcgctaaaccccaacagggtgactttcaaggccaacaggcctttcctggtttttataagagaagttcccctgaacactattatcttcatgggcagagtagccaacccttgtgttaagtaaaatgttcttattctttgcacctcttcctatttttggtttgtgaacagaagtaaaaataaatacaaactacttccatctcaaaaaaaaaaaaaaaaaaaaaaaaaaaaaaaaaaaaaaaaaaa

SEQ ID NO: 24 - DNA, 5195 bp, Artificial
  Synthetic
  misc_feature: Predicted druggable chimeras
  misc_feature: LENG8/RTF1
tgagagcatgtcctaccaggctccccctcagcagctgccgtcggctcagccccctcagccctcaaatcccccacatggggctcacacgctgaacagtggccctcagcctgggacagctccagccacacagcacagccaggcggggcccgccacgggccaggcctatgggccacacacctacaccgaacctgccaagcccaagaagggccaacagctgtggaaccgcatgaaacccgcccctgggactggaggtctcaagttcaacatccagaagcgaccctttgctgttaccacccagagctttggctccaacgcagagggccagcacagtggttttggcccccagcccaaccctgagaaagttcagaaccacagcgggtcctctgcccgggggaacctgtctgggaagccggatgactggccccaggacatgaaagagtatgtggagcgctgcttcaccgcctgtgagtcggaggaggacaaggaccgcacggaaaagctgctcaaggaggtgctgcaggcgcggctgcaggacggctcggcctataccattgactggagccgggagcccttgccggggctgacccgggagcctgtggctgagagccctaagaagaagcggtgggaggccgctagcagccttcaccctcctagaggggcaggctcggcgacaaggggcgggggtgccccgtcccagcgagggacgcccggggctgggggtgccggtcgagcccggggcaacagcttcaccaagtttggcaaccgcaacgtcttcatgaaggacaacagctcttcttccagcacagactcccgctcccgctcctcctccaggtccccgacgcgccacttccgcagaagtgactcccactcagactccgacagctcctactcagggaatgagtgtcaccctgtgggccgcaggaacccgccccctaagggccggggcggtcgaggggcccatatggatcggggccgaggcagggcgcagcgtgggaagaggcacgatctggcgcccaccaagcgcagtcgaaagaagatggcggcgctggagtgtgaggacccggagcgagagctgaagaagcagaagcgggcagcccgcttccagcacggacactcccgccgcctgcgcctcgagcccctggtgctgcagatgagcagcctggagagcagtggggctgaccctgactggcaggagctgcagatcgtgggcacctgccctgacatcaccaagcactacctgcgcctcacctgtgcccccgacccgtccaccgtgcgccctgtggcagttttgaaaaagtcgctgtgcatggtcaagtgccactggaaagagaagcaggactacgcgtttgcctgcgagcagatgaagtcgatccggcaggatctgacggtgcagggcatccgcaccgagttcacggtggaggtgtacgagacccatgcccggatcgccttggagaagggtgaccatgaagagtttaaccagtgccagacgcagctcaagtcgctgtacgccgagaacttgcctggcaatgtgggcgagtttactgcctaccgaatcctctactacatcttcaccaagaactcgggagacatcaccacggagctggcatacctcacacgagaactgaaggcagatccttgcgtggcccacgccttggcattaaggacagcctgggccctgggcaactaccaccgctttttccggctctactgccatgcaccctgcatgtctggctacctcgtggacaagtttgcagatcgggagcgcaaggtcgccctcaaggccatgatcaaaacgtatgtggtgccaagctcccttctgcctttgctcttcccatccttccgcctcgcaccgcccctcagaccagctcctggccgcaggcctcccccagcccccaacccttgtcctggtccttgcttccccatcatctttctccattcagccctcccctctccagttcctcttgctctccttgttggtcacctctgtgttccgggtcactcctctccctctccccactgttcccagctcactgcctctggggcctcttctccaccccatctgtgtgtctcttcctcctgttctctcctgcctggaccccctagttcactccttgccctgggcttcctcagaaccctgaggtctctgctttctcagcttgtcgctgtgctcccaccatagagaccatctagacagcctctggtctaccaggacaaggcccagtcccactcagctcctttgagagcaccagaaacgcttagggagacacctgtgttgaggccacactgggcgcggtgccagaggcccctggtcaggccatgcccctgcagtgtccttcgttcactagacattgcgcctggcttgctgtgggtggggatgagttgcttgactcatgtttagacgcatggttctgtctggtgattgaggtgcccaggcgacgctgggcaatgtcaagagaggttttcgcttgtcacagcaaggggatgctcttggcatctagtgagtggaggccagggatgctgccctgccactgccattgggcctcagagctcagtcttgccaaggtggagaaattctggtcgaagaggttgaccggtagactgaagaggccttgagagcctggccaggtggctgtccacgtgaggtcatagtcacagcatggaggccttgagagcctggccaggtggccctccacgtgaggtcatggtcacagcatgggggccttgagtgcctggccaggtggccctccacgtgaggtcatggtcacagcatggaggccttgagagcctggccaggtggccctccacgtgaggttgtggtcacagcatgggggccttgagagcctggccaggtggccctccacgtgaggtcatggtcacagcatggaggccttgagagcctggccaggtggccctccgtgtgaggtcatggtcacagcatagtttggagctagggagggactgtcagcctggaggtttggagactcattctggaatctagtgtgggtcaagccaacttcagggagaggctgagccagggtaggagtcacaggagcagacgaggatgtggggtgccgtgcacagagctccatgaccagcttgggaagttagaaggaaggggaggcaggaggctgcttagtctgctgccatgatgggccccatgaatggtggctctcaagcttctgtgctacacaggggtgtgtggttgggcgagtgtccttgttaattgatacctaattggccttggttgggaacatagccatgagtgcccctcgtgggtggggcgccagtctgtcattgacctcattgtgtcagcaccttccccccaaggaggctgaccctgccctgccagctgagactgggagacggagtgggctctgatcccagggcttccaggaaagctctggctttggagcagaggcgggttctaggacttagtgcccctcactcggtgcctgggttcctgtgtgtgtggaaagggtagggctgcaccctgtggaagagggttcagggagctctgaggacctggctcgtgctagatgctcagtcagtagtgtgttttagcagcagccggaagtgactgctttcagtgtgtagtggtgagtgctagctggcacccctgcgcctggcttcccagacactgtaacgcagggcaggggaggcagcgagggctgggtccccacggtggccctggcagccccgccatccccatcccctgccacgtgctgctccctccatctggtccctgcccctcagccagcggaggagcccgctgggcccttccctgcccgtccccgctcctccctggcccccgcacaggtaagtgagggtgaggggggcacaggggactggggtcactaaccccccccgggccccccccgagcccctgaggtggggaggctgggaggcgggtggggagccagcaggcactgtgctgaggaaatcctgtgggcacccgaatgggggggcctccccgctcgtggggccctcccctgccctcccgcccgcccgcctcatcaccttctcctcttgtctccatagcttccgccctgcgctgccagtctcctacctgcaggccgagctggccttcgagggcgaggccgcctgccgggccttcctggagcccctgggcctggcctacacgggcccggacaactccagcatcgactgccgcctcagcctggcgcagctgtcagccttctgagcacccagcgaggaggggcgggggcaggggctgcagcccccagcgctgcctttgcggattctgtttttgagccgtggacttgggttgtaaatttatttgtggggagtgcgctccaggaagagccaccatccctgcccccgttttcccaccggggagtctgtacagagatttttctacgtttttattttttgcctcagagggatgggattggggaggaggggatgggcagcggagggttgggggcatggtctgcaggctcatctgtgtccgcctttcactccactaatgctgtctcagtgttttctctctctctctttcgagcttgcactccggtacccgacccggcgccctggcccatcccatgccggggggccagtggaaagaagacaggccgtccagcccgtgcccgcctgcggcgggggcacccagcaagcccgcccaccgcccgctgcctcacctgcttcgccacagaggacagcggcagcgacgagaacctggatcaggagctcttgtccctggcaaagcgaaagcgcagtgactctgaggagaaggagccgcctgtgagtcagcctgcagcctcgtcagactcggagacgtctgacagtgacgatgagtggacatttgggagcaataaaaataagaagaaaggaaaagccagaaaaatagagaagaaaggaaccatgaagaaacaggccaacaaaactgcctcctcaggcagttcagacaaagacagttcagctgagagctcagcccctgaggaaggtgaagtgtcagactctgacagcaacagctcctcttccagttcagattcagactcttcctcagaagatgaagagttccatgatggctatggagaagacctcatgggagatgaggaagacagggcccgtctggaacagatgacagagaaagagagagagcaagaactgttcaatcgcatagagaagagggaggtgttgaaaagaagatttgaaatcaagaaaaaactaaaaac

SEQ ID NO: 25 - DNA, 313 bp, Artificial
  Synthetic
  misc_feature: Predicted druggable chimera
  misc_feature: LOC100996842/RCC2
tttttttttttttttttgacaaaaaactcatggcttttaatcctggcaggacaaggaaaaccccgtcaaaaatttgcataaattttgctgctctcctgctggggggggggggggggcctttaataccccttcaccctaaacctctttcaaattctttttcctgaactggaagaccattcatatgcccacagggtcagcgccaagcacattcccaaccgggcaacttgtgtcctttctctaggagtgcacgacacccttcccccacaactccttgttttaaaggatttaacccattaggaaacccatttttcaa

SEQ ID NO: 26 - DNA, 2821 bp, Artificial
  Synthetic
  misc_feature: Predicted druggable chimera
  misc_feature: NEGR1/RBM3
cccacgcgtccgagccgcccgagaattagacacactccggacgcggccaaaagcaaccgagaggaggggaggcaaaaacaccgaaaaacaaaaagagagaaacaacacccaacaactggggtggggggaagaaagaaagaaaagaaacccacccacccaccaaaaaaaaaaaaaaaaaaaaaaaaaaaaaaaaaaaaatcctgtggcgcgccgcctggttcccgggaagactcgccagcaccagggggtgggggagtgcgagctgaaagctgctggagagtgagcagccctagcagggatggacatgatgctgttggtgcagggtgcttgttgctcgaaccagtggctggcggcggtgctcctcagcctgtgctgcctgctaccctcctgcctcccggctggacagagtgtggacttcccctgggcggccgtggacaacatgatggtcagaaaaggggacacggcggtgcttaggtgttatttggaagatggagcttcaaagggtgcctggctgaaccggtcaagtattatttttgcgggaggtgataagtggtcagtggatcctcgagtttcaatttcaacattgaataaaagggactacagcctccagatacagaatgtagatgtgacagatgatggcccatacacgtgttctgttcagactcaacatacacccagaacaatgcaggtgcatctaactgtgcaagttcctcctaagatatatgacatctcaaatgatatgaccgtcaatgaaggaaccaacgtcactcttacttgtttggccactgggaaaccagagccttccatttcttggcgacacatctccccatcagcaaaaccatttgaaaatggacaatatttggacatttatggaattacaagggaccaggctggggaatatgaatgcagtgcggaaaatgctgtgtcattcccagatgtgaggaaagtaaaagttgttgtcaactttgctcctactattcaggaaattaaatctggcaccgtgacccccggacgcagtggcctgataagatgtgaaggtgcaggtgtgccgcctccagcctttgaatggtacaaaggagagaagaagctcttcaatggccaacaaggaattattattcaaaattttagcacaagatccattctcactgttaccaacgtgacacaggagcacttcggcaattatacctgtgtggctgccaacaagctaggcacaaccaatgcgagcctgcctcttaaccctccaagtacagcccagtatggaattaccgggagcgctgatgttcttttctcctgctggtaccttgtgttgacactgtcctctttcaccagcatattctacctgaagaatgccattctacaataaattcaaagacccataaaaggcttttaaggattctctgaaagtgctgatggctggatccaatctggtacagtttgttaaaagcagcgtgggatataatcagcagtgcttacatggggatgatcgccttctgtagaattgctcattatgtaaatactttaattctactcttttttgattagctacattaccttgtgaagcagtacacattgtcctttttttaagacgtgaaagctctgaaattacttttagaggatattaattgtgatttcatgtttgtaatctacaacttttcaaaagcattcagtcatggtctgctaggttgcaggctgtagtttacaaaaacgaatattgcagtgaatatgtgattctttaaggctgcaatacaagcattcagttccctgtttcaataagagtcaatccacatttacaaagatgcatttttttcttttttgataaaaaagcaaataatattgccttcagattatttcttcaaaatataacacatatctagatttttctgcttgcatgatattcaggtttcaggaatgagccttgtaatataactggctgtgcagctctgcttctctttcctgtaagttcagcatgggtgtgccttcatacaataatatttttctctttgtctccaactaatataaaatgttttgctaaatcttacaatttgaaagtaaaaataaaccagagtgatcaagttaaaccatacactatctctaagtaacgaaggagctattggactgtaaaaatctcttcctgcactgacaatggggtttgagaattttgccccacactaactcagttcttgtgatgagagacaatttaataacagtatagtaaatataccatatgatttctttagttgtagctaaatgttagatccaccgtgggaaatcattccctttaaaatgacagcacagtccactcaaaggattgcctagcaatacagcatcttttcctttcactagtccaagccaaaaattttaagatgatttgtcagaaagggcacaaagtcctatcacctaatattacaagagttggtaagcgctcatcattaattttattttgtggcaggtattatgacagtcgacctggagggtatggatatggatatggacgttccagagactataatggcagaaaccagggtggttatgaccgctactcaggaggaaattacagagacaattatgacaactgaaatgagacatgcacataatatagatacacaaggaataatttctgatccaggatcgtccttccaaatggctgtatttataaaggtttttggagctgcactgaagcatcttattttatagtatatcaaccttttgtttttaaattgacctgccaaggtagctgaagaccttttagacagttccatctttttttttaaattttttctgcctatttaaagacaaattatgggacgtttgtcaaaaaaaa

SEQ ID NO: 27 - DNA, 2660 bp, Artificial
  Synthetic
  misc_feature: Predicted druggable chimera
  misc_feature: TTYH3/WDR34
ggcacgaggcgcacacctggcagcaaaagagaggccctgatgaggacggggaggaggaggccgctccagggccgcggcaggcgcacgacagcctctaccgcgtccacatgcccagcctgtacagctgtggcagcagctacggcagtgagaccagcatcccggccgcggcccacaccgtcagcaacgccccggtcactgagtacatgagccagaacgctaatttccagaacccccgctgtgagaacaccccactcattgggcgcgagtccccgccgccctcatacacctccagcatgagagccaaatacctcgccacgagccagcctcgccctgactccagcggcagccactagaccgcgcccggcagccacccaccccacgtgccaacttcccctccccgtgccagcactgccgcttccacctgggccacccaccggaccctcgcacgccgtgccaggcctgccccagacgcgtctgcaggccgcttgccctcctgtcccctccccgcaggggcacagtggagacgcaggggctctgggcccgtaccgccaactcgggtcacacctgaacgctgctgccagccgatgccccagccctgcacgccacccactatcccggcacgctccctctgcagatggtcgccgcacctacaagccctggccgcacccaacctgtgttgttgccgcccggcccttccctccacagctctccttcctcccgcccggcacttctgtggaccccttcttagttcacaggcacggctggggccgctctgtgctggcgcctgctggccactgagggacagggacacgtgccacctgctcatctctgccctgaggtcaccccgtggtccctccacgtgcccatctctctgcagtgccctcctcgcctgtgcagcccgcccacccacaggctcacccctcctgccggctgccagaggccccctccagcagggcctctctccgttgccccagcttcactctctccctcagcacctgccctgctggaggccccagccctccgtggacagcaggggccacgtggagcccgggccgcccggggccggggcggccagggccgctgcaggacgagaccctgggtgtggcgtccgtgccctcgcagtggagggccgtccagggcatccgcggggagacgaaaagttgccagacggccagcattgccactgccagtgcatccgcccaggccaggaatcatgtggacgcccaggtgcagacggaggcccccgtgcctgtcagcgtgcagcccccgtcccagtacgacatacccaggctcgcagcctttcttcggagagtggaggccatggtcatccgagagctgaacaagaattggcagagccacgcgtttgatggcttcgaggtgaactggaccgagcagcagcagatggtgtcttgtctgtataccctgggctacccgccagcccaagcgcagggtctgcatgtgaccagcatctcctggaactccactggctctgtggtggcctgtgcctacggccggctggaccatggggactggagcacgcttaagtccttcgtgtgtgcctggaacctggaccggcgagacctgcgtccccagcaaccgtcggccgtggtggaggtccccagcgctgtcctgtgtctggccttccaccccacgcagccctcccacgtcgcaggagggctgtacagtggtgaggtgttggtgtgggacctgagccgtcttgaggacccgctgctgtggcgcacaggcctgacggatgacacccacacagaccctgtgtcccaggtggtgtggctgcccgagcctgggcacagccaccgcttccaggtgctgagtgtggccactgacgggaaggtgctactctggcagggcatcggggtaggccagctgcagctcacagagggcttcgccctggtcatgcagcagctgccacggagcaccaagctcaagaagcatccccgcggggagaccgaggtgggcgccacggcagtggccttctccagctttgaccctaggctgttcattctgggcacggaaggcggcttcccgctcaagtgttccctggcagctggagaggcagccctcacgcggatgcccagctccgtgcccctgcgggccccagcacagtttaccttctccccccacggcggtcccatctactctgtgagctgttcccccttccacaggaatctcttcctgagcgctgggactgacgggcatgtccacctgtactccatgctgcaggcccctcccttgacttcgctgcagctctccctcaagtatgtttgctgtgcgctggtccccagtgcggcccttggtttttgcagctgcctctgggaaaggtgacgtgcagctgtttgatctccagaaaagctcccagaaacccacagttttgatcaagcaaacccaggatgaaagccctgtctactgtctggagttcaacagccagcagactcagctcttggctgcgggcgatgcccagggcacagtgaaggtgtggcagctgagcacagagttcacggaacaagggccccgggaagctgaggacctggactgcctggcagcagaggtggcggcctgaggggtcccgggaggcgggtgcaagccttcgctgtgccgagccttgtgtttctgacgcaagccaaatgaagaaaagcaaagct

SEQ ID NO: 28 - DNA, 2660 bp, Artificial
  Synthetic
  misc_feature: Predicted druggable chimera
  misc_feature: RACK1/ATP1A1_Junction2
ggcacgagggcgacaggcacgcagcctactaggtgtggcggcgaccctggccccgggttcccgtggctaccgggcgcggccgcccccgcgccgcaggccgggaccccggtggccagaccccgaggacctcctgaccccgcggtggcagctgggaccgcgctacgcggctaagcagttcgcgcgttacggcgccgcctccggggtggtccccggttcgttatggccgtcgccggagcagctgcgggagctggaggccgaagaacgcgaatggtacccgagcctggcgaccatgcaggagtcgctgcgggtgaagcagctggccgaagagcagaagcgtcgggagagggagcagcacatcgcagagtgcatggccaagatgccacagatgattgtgaactggcagcagcagcagcgggagaactgggagaaggcccaggctgacaaggagaggagggcccgactgcaggctgaggcccaggagctcctgggctaccaggtggacccaaggagtgcccgcttccaggagctgctccaggacctagagaagaaggagcgcaagcgcctcaaggaggaaaaacagaaacggaagaaggaggcgcgagctgctgcattggctgcagctgtggctcaagacccagcagcctctggggcaccccagctcctgaggctttgtcccttcccaataaagcctgctacctggcagtacccctgaaaaaaaaaaaaaaaaaaaaaaaaaaaaaaaaaaaaaaaaactcgagttttttttttttttttttcagaaaagccagttttttttttatttgtaaagctctgccataaacttctagcgtgtgccaatggtcacctgccacactcgcaccaggttgtccgtgtagccagcaaacagagtctggccatcagcagaccaggccagggaggtgcactggggtggttctgccttgctgctggtactgataacttcttgcttcagttcatctacaatgatctttccctctaaatcccagatcttgatgctggggcctgtggcagcacacagccagtagcggttagggctgaagcacagggcgttgatgatgtccccaccatctagcgtgtaaaggtgtttgccttcgttgagatcccataacatggcctggccatccttgcctccagaagcacagagggatccatctggagagacagtcaccgtgttcagatagcctgtgtggccaatgtggttggtcttcagcttgcagttagccaggttccataccttgaccagcttgtcccagccacaggagacgatgatagggttgctgctgttgggcgagaagcggacacaagacacccactctgagtggctctcatcctggacagtgtatttgcacacacccagggtattccatagcttgatggttttatctcgagatccagagacaatctgccggttgtcagaggagaaggccacactcagcacatccttggtatggcccacaaatcgcctcgtggtggtgcccgttgtgagatcccagaggcgcagggttccatcccaggagcctgagagggcaaactggccatctgaggagataaccacatcactaacaaagtgggagtgaccccgcagagcacgctgtggaattccatagttggtctcatccctggtcagtttccacatgatgatggtcttatctcgagaggcggagaggatcatgtccgggaactgcggggtagtagcgatctgggttacccagccgttgtggcccttgagggtgccacgaagggtcatctgctcagtcatggcggcggcgagagcgtgtgtcgctgcagcgacgaggatggcactggatggcttagagaaactagcaccacaacctctcctgccgccgccttgcctgagaacggcttcctcccaattcacctgttgggcctccgagtggactgggatgaccgctggatcaacgatgtggaagacagctacgggcagcagtggacctatgagcagaggaaaatcgtggagttcacctgccacacagccttcttcgtcagtatcgtggtggtgcagtgggccgacttggtcatctgtaagaccaggaggaattcggtcttccagcaggggatgaagaacaagatcttgatatttggcctctttgaagagacagccctggctgctttcctttcctactgccctggaatgggtgttgctcttaggatgtatcccctcaaacctacctggtggttctgtgccttcccctactctcttctcatcttcgtatatgacgaagtcagaaaactcatcatcaggcgacgccctggcggctgggtggagaaggaaacctactattagccccccgtcctgcacgccgtggagcatcaggccacacactctgcatccgacacccaccccctctttgtgtacttcagtcttggagtttggaactctaccctggtaggaaagcaccgcagcatgtggggaagcaagacgtcctggaatgaagcatgtagctctatggggggaggggggagggctgcctgaaaaccatccatctgtggaaatgacagcggggaaggtttttatgtgcctttttgtttttgtaaaaaaggaacacccggaaagactgaaagaatacattttatatctggatttttacaaataaagatggctattataatgaaaaaaaaaa

SEQ ID NO: 29 - DNA, 3834 bp, Artificial
  Synthetic
  misc_feature: Predicted druggable chimera
  misc_feature: ANKRD52/RPS21
ccacgcgtccgaatgagcatgtgctttcagctgggtttgacatcaatacacctgacaaccttggccgtacctgtcttcatgctgctgcttccggagggaatgttgaatgtcttaatttgctgttgagcagtggagctgacttgaggaggagggacaaatttggcaggaccccactgcactatgcagctgctaacggtagctaccagtgtgcagtaacattggtgactgctggggcaggtgtcaacgaggccgactgtaaaggctgctctcccctccactacgctgccgcttctgacacttacaggagagcggaaccccatacaccttccagccatgatgccgaagaggacgagccactgaaggagtcccgcaggaaggaggccttcttctgtctggagttcttactggataacggtgcagacccctccctgcgggacaggcagggctacacagctgtgcactatgcagccgcctatggcaacagacagaacctcgaactgctcttagaaatgtcctttaactgcctggaggatgtggagagcaccattccagtcagccctttgcacttagctgcctacaacggtcactgtgaagccttgaagacgctggcggagacgctggtgaatctggacgtaagggaccacaagggccggaccgcactcttcctggccacggagcgcggctctactgagtgtgtggaggtgcttacagcccacggcgcctctgccctcatcaaggagcgcaagcgcaagtggacacccctgcacgctgctgctgcctctggccacactgactccctgcacttgctgatcgacagtggggaacgagctgacatcacagatgtcatggatgcctatggacagaccccactgatgctggccatcatgaatggccatgtggactgtgtacatctgctgctagagaaaggatccacagctgatgctgctgacctccggggccgcactgccctccaccgcggggcagtgactggctgtgaggactgcctggctgccctgctggaccacgacgcatttgtgctgtgccgagactttaagggccgcacgcccattcacctggcctcagcctgtggccacactgcagtactgcggaccctgctgcaggctgccctttccacagatcccctggatgccggggtggattacagcggatactcgcccatgcactgggcctcctacactggacatgaagattgtctggagttgttacttgaacacagcccgttttcgtacctggaaggaaaccccttcactcctttgcactgtgcagtgattaataaccaagacagcaccacagagatgctactgggagctctgggtgccaagattgtgaacagccgagatgccaaaggacggaccccccttcacgccgctgccttcgcggacaatgtctctgggctccggatgctgctgcagcatcaagctgaggtgaacgccactgaccacactggccgcactgcgctcatgacggcggctgagaacgggcagaccgctgctgtggaatttctgctgtatcgagggaaggcagaccttactgtgttggatgagaacaagaacacggccctccacttggcttgtagcaagggccatgagaaatgtgccctcatgatcctggcagaaacccaagaccttggccttatcaatgctaccaacagtgcgctgcagatgccactccacattgctgcccggaatggtctagcttctgtggtacaggccctgctgagtcatggggccacagtgctggctgtggatgaagaaggtcacaccccagcactggcctgtgcccccaacaaagatgtggcagactgcctggccttgatcctttccaccatgaagcctttcccacccaaggacgccgtcagtcctttcagcttcagcctgctcaagaactgcagcattgcagccgccaagacggtgggtggctgcggcgccctgccccatggggcctcctgcccctacagccaggagcggcccggcgccattgggttagatggctgctactctgagtagccccctccagtgtccctcccccgccggtggcttgatatctaattctatttatttagaaaaagtctaaacatttagggcactttaaaggagaacacgactgggtggagggggcggaggggaaggaagccctggggagcagctgctcacccctttgccacaccatcttggcctggcaggggtctgggactgacagggagcaccccaggcccttggtacccccagggcgaccccttctgccaagtgtcccaaaatgattgctaaatgcctggctcccccactctttgactccatctcttggttccctctttctgctgccagctcccccgactcttccctggggactcctctctgtgtcccccttctcccctgcccctactgccaggcagatcccctcttcttccatacccatcactgcctccctgctcggccggtccctccatccccgcagcagtgagaagccttaatttctggtactgtgtgagcactctgggggtgtccccctccccccttcaggggcagctagaggatgaggggggaggatatttagcactggggcctggagctttttccccacttctgtaccctatcacccctaaagttcaaatgcaaaacacttgaagcagcaactactgtacctgggccccaatcctgcactctcccctggctcctcatatgcaaatcaagggctggttctgcccccacagcctgccccctccccattccctcccactcctagcctggcccctaaccacgcactttaaccttgcttctttcttttatcttcttttgggagggcagagcctgtggtcattcctgccctggctctccttcccttgaactttgaggcttgtcatgaatttttaagtaagcgttttatcctttaggggaagaaacaaattaatttcctgcccatttcaaaaccacagccctagtatgtccactgtgtttcaggcatgtgtttgcatgtatatggagggagggaccatgttcttcctcctgtgccgccaagcccatagtttatctgtgctccctatctgcctccaccttccgttatgtccaagagtacccactgccccagtcactcctgatctgaagccaaagatggtaggaggggcagggccgacaagggaccgtggctactcgggacccaggcttcccctccagtgtgaggaagaagctctgacctagagagagatgaataggtactggggcttggtccctgccttcccagcagggaggaaagatgggattgagctgggggctgcactgataagatttctaaagtcgcaccccaaaagccaaactgggctggagtgaagagggggcagttttctctctaaatgtagcattgaatttggccctggtccctttaagtgctctgtccacccatctcctagtgcagcctcctggacagttggacccactcctgcagcccagcctcgaaatgcagaacgacgccggcgagttcgtggacctgtacgtgccgcggaaatgctccgctagcaatcgcatcatcggtgccaaggaccacgcatccatccagatgaacgtggccgaggttgacaaggtcacaggcaggtttaatggccagtttaaaacttatgctatctgcggggccattcgtaggatgggtgagtcagatgattccattctccgattggccaaggccgatggcatcgtctcaaagaacttttgactggagagaatcacagatgtggaatatttgtcataaataaataatgaaaacctaaaaaaaaaaaaaaaaaaaaaaaaaaaaaaaaaaaaaaaaaaaaaaaaaaaaaaaaaa

SEQ ID NO: 30 - DNA, 2537 bp, Artificial
  Synthetic
  misc_feature: Predicted druggable chimeras
  misc_feature: ZFYVE16/GOT1
gtgcaggtggtggcgaagcgctcctccgaaaggtttcggaagctggtggtagctctgaagataacgctgcgttagggcatactgcggcggaggatggaactccgattgaaagcagttgctggagtggagcacgaatttcaacaagccgcatgttgaagtgtgaggcgtgaaagggtatgtctgatatttgctttaaaatgctccagcaaagaaattaagggatggatgaagcaaaagagccaggtatggtggctcatgcctctaatctcagcactttgggaggccgaagcaggcagatcacctgaggtcaggagtttgagaccatcctgaccaacatggtgaaactcgtctctactacaaacataaaaaaattagctgggcgtggtaccacgcacctgtaatcccagctactcctctggaggctcagacaggagaactgcttgaaccgggaggcggaggttgcagtgagctgagatcgcaccactccattccagcctgggcaacagagtgagactccatctcataaaaaaaaaaaaagtaaaaaaaaacggccagacctgacctgccagctccgggcgtggggtgaaatctcttgattcctagtctctcgatatggcacctccgtcagtctttgccgaggttccgcaggcccagcctgtcctggtcttcaagctcactgccgacttcagggaggatccggacccccgcaaggtcaacctgggagtgggagcatatcgcacggatgactgccatccctgggttttgccagtagtgaagaaagtggagcagaagattgctaatgacaatagcctaaatcacgagtatctgccaatcctgggcctggctgagttccggagctgtgcttctcgtcttgcccttggggatgacagcccagcactcaaggagaagcgggtaggaggtgtgcaatctttggggggaacaggtgcacttcgaattggagctgatttcttagcgcgttggtacaatggaacaaacaacaagaacacacctgtctatgtgtcctcaccaacctgggagaatcacaatgctgtgttttccgctgctggttttaaagacattcggtcctatcgctactgggatgcagagaagagaggattggacctccagggcttcctgaatgatctggagaatgctcctgagttctccattgttgtcctccacgcctgtgcacacaacccaactgggattgacccaactccggagcagtggaagcagattgcttctgtcatgaagcaccggtttctgttccccttctttgactcagcctatcagggcttcgcatctggaaacctggagagagatgcctgggccattcgctattttgtgtctgaaggcttcgagttcttctgtgcccagtccttctccaagaacttcgggctctacaatgagagagtcgggaatctgactgtggttggaaaagaacctgagagcatcctgcaagtcctttcccagatggagaagatcgtgcggattacttggtccaatccccccgcccagggagcacgaattgtggccagcaccctctctaaccctgagctctttgaggaatggacaggtaatgtgaagacaatggctgaccggattctgaccatgagatctgaactcagggcacgactagaagccctcaaaacccctgggacctggaaccacatcactgatcaaattggcatgttcagcttcactgggttgaaccccaagcaggttgagtatctggtcaatgaaaagcacatctacctgctgccaagtggtcgaatcaacgtgagtggcttaaccaccaaaaatctagattacgtggccacctccatccatgaagcagtcaccaaaatccagtgaagaaacaccacccgtccagtaccaccaaagtagttctctgtcatgtgtgttccctgcctgcacaaacctacatgtacataccatggattagagacacttgcaggactgaaaggctgctctggtgaggcagcctctgtttaaaccggccccacatgaagagaacatcccttgagacgaatttggagactgggattagagcctttggaggtcaaagcaaattaagatttttatttaagaataaaagagtactttgatcatgagacataggtatcttgtccctctcactaaaaaggagtgttgtgtgtggcggccacgtgcttctatgtggtgtttgactctgtacaaattctagtcccaaagatcaagttgtctgaaggagccaaagtgtgaatgtgggtgtcggctgcggcattaaattcatcatctcaacccagagtgtctggtctccctgctctttctgcatggttgtgtccctagtcctaagctttggttctttagggtgactgtggtaagaaggatatttaatcatgacatgcacggacacgtacatatttaactgaaacaagttttaccaaacagtatttactcgtgatgtgcgtagtgcattctgatatttttgagccattctattgtgttctacttcacctaaaaaaataaaataaaaatgttgatcaagaaaaaaa

SEQ ID NO: 31 - DNA, 439 bp, Artificial
  Synthetic
  misc_feature: Predicted druggable chimera
  misc_feature: RNA45SN1/RNA45SN2
attaaggactatgaatgcttggcagcacaatacagttcctgtggtaacttttctgacacctcctgcttaaaacccaaaaggtcagaaggatcgtgaggccccgctttcacggtctgtattcgtactgaaaatcaagatcaagcgagcttttgcccttctgctccacgggaggtttctgtcctccctgagctcgccttaggacacctgcgttaccgtttgacaggtgtaccgcccttcacaaagaaaagagaactctccccgggggctcgcccccccgcctcacctctcatgtctcttcaccgtgccagactagagtcaagctcaacagggtcttctttccccgctgattccgccaagcccgttcccttggctgtggtttcgctggatagtaggtagggacagtgggaatctcgttcatccattcatgcgcgtcactaat

SEQ ID NO: 32 - DNA, 511 bp, Artificial
  Synthetic
  misc_feature: Predicted druggable chimera
  misc_feature: SLC25A44/RIPK2
ctgatgttggagcggccgcagaatcaagatttttttttttttttttttttcgggggctcaataaaaacttttaattacatttcagagacttcgtacagtgcaacagtgaatattcactgttaattttcacaagagtccatttcatcaaacgttcagagagtctgcctgtccccatttttaacctcagccttccctactgtcaccaacaaccaagctaaataaagtcaacagcctgaaaaaaaaaaaaaaaaaaaaaaaaaaaaaaaaaaaaaaaaaaaaaaaaaaaaaaaaaaattcttgattcggcggccgcaagcttattccctttagggggggttaattttaccttggaacgggccgtcgttttaaaacgtcgtgacggggaaaaccctggggttacccaacttaatcgccttgcagaaaatccccctttcgcaaggtggggtaatagcgaaaaggcccgcaccgttcgcccttccaaaaatttgcccaccctgaatggcaaagggga

SEQ ID NO: 33 - DNA, 548 bp, Artificial
  Synthetic
  misc_feature: Predicted druggable chimeras
  misc_feature: EMP2/EMP2
gcgcttattggatgtctactaattttgttgttgttattgttctcgaataactggtaataagcaccaagtgagggctgattccatctacaggtaagaagcttggatgttttctccctgattttgtcatttctttccaacttcccaaatgcatgtcatacagcagggaagaggcatctctcatgtctcaaaaaagcttaatggcacagatggatgtgggcactgaatgttgtacagtagtggcaaccagccgggaaaatccataggatcatttcagaccgcattcaacacagatactcatgtagccccgacgtttcttgtgaagggaattgatgtattgcttgagggggtcgatctctggttctaaggtgttttgtttgtttccatcgtgactaccagaaaattatagaaggccagacgcagtggctcatggcctagtggaggagacagattaaaacttaattagaaaaactggctgggtacagtggctcatgcttgtaatcccagcactttgggaggctgaggcgggtggatcacctgaggtcaggaat

SEQ ID NO: 34 - DNA, 664 bp, Artificial
  Synthetic
  misc_feature: Predicted druggable chimeras
  misc_feature: TPX2/ERGIC1
ctaggtgagccgtgggaagaaaagagggagcagctagggcgcgggtctccctcctcccggagtttggaacggctgaagttcaccttccagcccctagcgccgttcgcgccgctaggcctggcttctgaggcggttgcggtgctcggtcgccgcctaggcggggcagggtgcgagcaggggcttcgggccacgcttctcttggcgacaggattttgctgtgaagtccgtccgggaaacggaggaaaaaaagagttgcgggaggctgtcggctaataacggttcttgatacatatttgccagacttcaagatttcagaaaaggggtgaaagagaagattgcaactttgagtcagacctgtaggcctgatagactgattaaaccacagaaggtgacctgctgagaaaagtggtacaaatactgggaaaaacctgctcttctgcgttaagtgggagacaatgtcacaagttaaaagctcttattcctatgatgccccctcggatttcatcaatttttcatccttggatgatgaaggagatactcaaaacatagattcatggtttgaggagaaggccaatttggagaataagttactggggatggaaatgtgtggcatgcacatgagttgaaattcttttatgcatttttttgaagaaaaaaaaaaaaa

SEQ ID NO: 35 - DNA, 500 bp, Artificial
  Synthetic
  misc_feature: Predicted druggable chimeras
  misc_feature: MIR44352HG/TRIOBP
ccattctgatgttggagcggccgcgataaggccatttttttttttttttttttttttttttttttttttttttttttttttttttttctttagttttgcttttaatgaaggacaagggattaagacacacagagactggccagacaaatgggaaaccgaccggaccagccgccctccttccctcctggatggaagtaaaaagccaagctttctccccaccctctgtgggccacacgtgcacttgcacccaccacacacacacacacacacacacacacacacacagacacacagacacatacgcacacacgtgcacacatgtacacacggatacacacacacacacacacacacacacactgcatatctaagcacgcccctcgcactgggtctcaccttgcaccttcttcaggattttatatgtgaagagatttttatatagatttttttcctttttttccaaaacactttatactttaaaaaaaaaaaaaaaaaatggc

SEQ ID NO: 36 - DNA, 2674 bp, Artificial
  Synthetic
  misc_feature: Predicted druggable chimera
  misc_feature: PRB2/C1S
cacagagttgggagtgactccagagcctcctgcaagatgctgttgattctgctgtcagtggccttgctggccctgagctcagctcagaacttaaatgaagatgtcagccaggaagaatctccctccctaatagcaggaaatccacaaggagcacccccacaaggaggcaacaaacctcaaggtcccccatctcctccaggaaagccacaaggaccacccccacaaggaggcaaccagcctcaaggtcccccacctcctccaggaaagccacaaggaccacccccacaaggaggcaacaaacctcaaggtcccccacctccaggaaagccacaaggaccacccccacaaggagacaagtcccgaagtccccgatctcctccaggaaagccacaaggaccacccccacaaggaggcaaccagcctcaaggtcctccacctcctccaggaaagccacaaggaccacccccacaaggaggcaacaaacctcaaggtcccccacctccagaaagccaggaccaagagacaggcagctcaccagggtggacaaatcgccagagatgtggtgcattgtcctgttttcacttttggcatgggtttatgctgagcctaccatgtatggggagatcctcccctaactatcctcaggcatatcccagtgaggtagagaaatcttgggacatagaagttcctgaagggtatgggattcacctctacttcacccatctggacattgagctgtcagagaactgtgcgtatgactcagtgcagataatctcaggagacactgaagaagggaggctctgtggacagaggagcagtaacaatccccactctccaattgtggaagagttccaagtcccatacaacaaactccaggtgatctttaagtcagacttttccaatgaagagcgttttacggggtttgctgcatactatgttgccacagacataaatgaatgcacagattttgtagatgtcccttgtagccacttctgcaacaatttcattggtggttacttctgctcctgccccccggaatatttcctccatgatgacatgaagaattgcggagttaattgcagtggggatgtattcactgcactgattggggagattgcaagtcccaattatcccaaaccatatccagagaactcaaggtgtgaataccagatccggttggagaaagggttccaagtggtggtgaccttgcggagagaagattttgatgtggaagcagctgactcagcgggaaactgccttgacagtttagtttttgttgcaggagatcggcaatttggtccttactgtggtcatggattccctgggcctctaaatattgaaaccaagagtaatgctcttgatatcatcttccaaactgatctaacagggcaaaaaaagggctggaaacttcgctatcatggagatccaatgccctgccctaaggaagacactcccaattctgtttgggagcctgcgaaggcaaaatatgtctttagagatgtggtccagagaaccctttgaagaaaaacagaggataattggaggatccgatgcagatattaaaaacttcccctggcaagtcttctttgacaacccatgggctggtggagcgctcattaatgagtactgggtgctgacggctgctcatgttgtggagggaaacagggagccaacaatgtatgttgggtccacctcagtgcagacctcacggctggcaaaatccaagatgctcactcctgagcatgtgtttattcatccgggatggaagctgctggaagtcccagaaggacgaaccaattttgataatgacattgcactggtgcggctgaaagacccagtgaaaatgggacccaccgtctctcccatctgcctaccaggcacctcttccgactacaacctcatggatggggacctgggactgatctcaggctggggccgaacagagaagagagatcgtgctgttcgcctcaaggcggcaaggttacctgtagctcctttaagaaaatgcaaagaagtgaaagtggagaaacccacagcagatgcagaggcctatgttttcactcctaacatgatctgtgctggaggagagaagggcatggatagctgtaaaggggacagtggtggggcctttgctgtacaggatcccaatgacaagaccaaattctacgcagctggcctggtgtcctgggggccccagtgtgggacctatgggctctacacacgggtaaagaactatgttgactggataatgaagactatgcaggaaaatagcaccccccgtgaggactaatccagatacatcccaccagcctctccaagggtggtgaccaatgcattaccttctgttccttatgatattctcattatttcatcatgactgaaagaagacacgagcgaatgatttaaatagaacttgattgttgagacgccttgctagaggtagagtttgatcatagaattgtgctggtcatacatttgtggtctgactccttggggtcctttccccggagtacctattgtagataacactatgggtggggcactcctttcttgcactattccacagggataccttaattctttgtttcctctttacctgttcaaaattccatttacttgatcattctcagtatccactgtctatgtacaataaaggatgtttataagcaaaaaa

SEQ ID NO: 37 - DNA, 1470 bp, Artificial
  Synthetic
  misc_feature: Predicted druggable chimeras
  misc_feature: SUMF2/RILPL1
ggcggcgcagcgcggcagtcctgatggcccggcatgggttaccgctgctgcccctgctgtcgctcctggtcggcgcgtggctcaagctaggaaatggacaggctattagcatggtccaactgcagggtgggagattcctgatgggaacaaattctccagacagcagagatggtgaagggcctgtgcgggaggcgacagtgaaaccctttgccatcgacatatttcctgtcaccaacaaagatttcagggattttgtcagggagaaaaagtatcggacagaagctgagatgtttggatggagctttgtctttgaggactttgtctctgatgagctgagaaacaaagccacccagccaatgaagcctgcaggtcctggctctggcatccgagagagactggagcacccagtgttacacgtgagctggaatgacgcccgtgcctactgtgcttggcggggaaaacgactgcccacggaggaagagtgggagtttgccgcccgagggggcttgaagggtcaagtttacccatgggggaactggttccagccaaaccgcaccaacctgtggcagggaaagttccccaagggagacaaagctgaggatggcttccatggagtctccccagtgaatgctttccccgcccagaacaactacgggctctatgacctcctggggaacgtgtgggagtggacagcatcaccgtaccaggctgctgagcaggacatgcgcgtcctccggggggcatcctggatcgacacagctgatggctctgccaatcaccgggcccgggtcaccaccaggatgggcaacactccagattcagcctcagacaacctcggtttccgctgtgctgcagacgcaggccggccgccaggggagctgtaagcagccgggtggtgacaaggagaaaagccttctagggtcactgtcattccctggccatgttgcaaacagcgcaattccaagctcgagagcttcagcctcaggaaagaacttccccttccctgtctcccatccctctgtggcaggcgcctctcaccagggcaggagaggactcagcctcctgtgttttggagaaggggcccaatgtgtgttgacgatggctgggggccaggtgtttctgttagaggccaagtattattgacacaggattgcaaacacacaaacagttggaacagagcactctgaaaggccattttttaagcattttaaaatctattctctccccctttctccctggatgattcaggaagctgacattgtttcctcaaggcagaattttcctggttctgttttctcagccagttgctgtggaaggagaatgctttctttgtggcctcatctgtggtttcgtgtccctctgaaggaaactagtttccactgtgtaacaggccagacttctgcgttaacttcagtaacacaagcttctttaagccaaatacatcacttgccactaaaaaaaaaaaaaaaa

SEQ ID NO: 38 - DNA, 1190 bp, Artificial
  Synthetic
  misc_feature: Predicted druggable chimeras
  misc_feature: PARP10/PARP10
gtggcacgatctcggctcacggcaagctccgcctcccgggttcacgccattctcctgcctcagcctcccaagtagctgggactacaggcgcctgccaccacgcccggctaattttttaaaaaatatttttagtggagacggggtttcaccatgttagccaggatggtctcgatctactgacctcgtgatccgcccgcctcggcctcccaaagtgctgggattacaggcgtgagccaccgcgcccggccttgcacggggcactctcaactcttgatgcccaaagttgtgcgtggggccagcagcatcaccctccaccaggagctggtcagaaatgcaaaaagaacctctggctgtccccagacctcaccaccatcatcaccaccatcacaccatcaccatgaccctcaccaccatcatcaccaccatcatcaccatcaccccatcaccatcaccaccaccatcacaccatcaccatgaccctcaccactcactgtcatcaccaccaccatcaccatcaccatcatcaccaccaccttcaccactcactgtcgtcaccaccatcatcaccaccaccttcaccactcactgtcgtcaccaccatcatcaccaccaccttcaccactcactgtcgtcaccaccatcatcaccaccaccttcaccactcactgtcgtcaccaccatcatcaccatcaccttcaccactcactgtcgtcaccaccatcatcaccaccaccttcaccactcactgtcgtcaccaccatcatcaccaccaccttcaccactcactgtcgtcaccaccatcatcaccatcaccttcaccactcactgtcgtcaccaccatcatcaccaccaccttcaccactcactgtcgtcaccaccatcatcaccatcaccttcaccactcactgtcgtcaccaccatcaccaccaccttcaccactcactgtcgtcaccaccatcatcaccaccaccttcaccactcactgtcgtcaccaccatcatcaccaccaccttcaccactcactgtcgtcaccaccatcatcaccatcaccttcaccactcactgtcgtcaccaccatcatcaccatcaccttcaccactcactgtcgtcaccaccatcaccaccaccactaccaccaccaccgtggccaccatcatcaccaccattagcatcaccactgtcaccgtcag